SEQUENCE LISTING (1 sequence)

SEQ ID NO: 1 - protein, 56 aa, HOMO SAPIENS
GSHMAEPQRHKILCVCCKCDGRIELTVESSAEDLRTLQQLFLSTLSFVCPWCATNQ

(19) United States
(12) Patent Application Publication
(10) Pub. No.: US 2016/0146801 A1
(43) Pub. Date: May 26, 2016

(54) NOVEL BIOMARKER DETECTION WITH MOLECULAR IMPRINTS

(71) Applicant: University of Houston System, Houston, TX (US)

(72) Inventors: Dong CAI, Houston, TX (US); Zhifeng REN, Houston, TX (US)

(21) Appl. No.: 14/817,879

(22) Filed: Aug. 4, 2015

Related U.S. Application Data
(60) Provisional application No. 62/032,973, filed on Aug. 4, 2014.

(51) Int. Cl. G01N 33/543; C07K 14/47

(52) U.S. Cl. CPC G01N 33/54373; C07K 14/47

(57) ABSTRACT

A bio-recognition system is described, where a bio-recognition nanosensor system uses an electropolymerization to produce protein imprints for biomarker recognition. Methods of making a bio-recognition sensor include forming a electropolymer coating on an electrode, binding a target biological structure to the coating to form an imprint, removing the biological structure from the coating, and forming a template that binds a specific biological structure.

### CROSS-REFERENCE TO RELATED APPLICATIONS

This application claims the priority of U.S. Provisional application Ser. No. 62/032,973, filed on Aug. 4, 2014 and incorporated herein in its entirety by reference.

### STATEMENT REGARDING FEDERALLY SPONSORED RESEARCH OR DEVELOPMENT

This invention was made with government support under Grant No. 5R21CA137681-02 awarded by the National Cancer Institute, and Grant No. DTRA HDTRA1-10-1-0001 awarded by the United States Department of Defense. The United States government has certain rights in the invention.

### BACKGROUND

1. Field of the Disclosure

This disclosure generally relates to a bio-recognition system, more specifically the disclosure relates to a bio-recognition nanosensor system using electropolymerization to produce protein imprints for biomarker recognition.

2. Background of the Technology

Biomarker proteins are molecules for specifically identifying cancers or other diseases.¹ For example, prostate-specific antigen (PSA), carcinoma antigen 125 (CA 125), cancer antigen 15-3 (CA 15-3), and carcinoembryonic antigen (CEA) are used for diagnose prostate cancer, ovarian cancer, breast cancer, and colon cancer respectively.² The early detection of low-concentration cancer biomarkers is crucial for the outcome of the treatment and 5-year survival rate of patients.^{3,4} The limit-of-detection (LOD) determines the capability of a biosensing technique to diagnose early stage cancers. In general, LOD can be divided into two components, i.e. the instrumental detection limit (IDL) and the method detection limit (MDL).⁵ For an immunosensor based detection system IDL characterizes the performance of transducer and signal amplification electronics, and MDL is related to the bio-recognition of the biomarker. Various micro and nanomaterials like gold nanomaterials^{6,7}, carbon nanotubes,⁸ nanoparticles,⁹ nanowire transistors¹⁰, enzyme-labeled beads^{11,12} and microarrays¹³ have been used in the sensor and demonstrated eminent capability to reduce the IDL. Monoclonal antibodies provide bio-recognition that supports the sensitivity and low MDL, but there are concerns stemming from their cross-reactivity among a complex body of other protein interference in clinic samples, which can introduce noise and significantly compromises the detection performance.

A Molecular imprint (MI) technology has been examined as a substitution for antibody/protein recognition.¹⁴ MIs with a number of transducer platforms for the preparation of MI-based protein sensors have been formed.¹⁵ Self-assembled monolayers have been imprinted to fabricate sensing elements to detect carcinoembryonic antigen (CEA) colon cancer biomarker.¹⁶ Electrodeposition of gold microdendrites and prussian blue particles on the glassy carbon surface derived a protein imprinted electrochemical sensor with better LOD.¹⁷ Carbon nanotube (CNT) array has also been used as an electrode, wherein Polyphenol (PPn) nanocoating was electropolymerized on the CNTs, and protein imprints were then fabricated in it.

Further, the detection of human papillomavirus E7 biomarker protein (E7) was conducted with the electrochemical impedance spectroscopy (EIS). The LOD was less than 1 ng/L.¹⁸ Similarly by the electropolymerization of PPn, the CA 125 biomarker was imprinted on a gold nanowire array. The LOD of CA 125 measurement with EIS was observed at 0.5 U mL¹⁹ which is lower than the LOD of ELISA, and a tenth of the level required for early diagnosis of cancer, when biomarkers are released into the blood stream from a tumor less than 4 mm in diameter.²⁰ Such improvements in molecular recognition/detection may be ascribed to the combination of a highly sensitive nanosensor and high quality MI, which contributes to the decrease of both IDL and MDL. Even though these data provide improvements in the design of biosensors, the existing imprint technology cannot recognize protein biomarkers with the required affinity for such early detection and diagnosis of disease biomarkers; recognize a precise mutation; or posttranslational modifications (PTM's) in proteins.

Therefore, there is a recognized need in the field for a bio-recognition system that can identify unique and known protein structural motifs (and other species) at extremely low molecular concentrations because they are unrecognizable with conventional methodologies. Such a bio-recognition system would have an impact on cancer detection and diagnosis by improving early detection and therefore improving treatment outcome by identifying cancer biomarkers and their profiles at early stages of the disease.

The bio-recognition system presented herein addresses such issues, whereby computational analysis of protein-polymer interactions and subsequent imprint design yields a physical imprint with a customized specificity for a particular target species.

### BRIEF SUMMARY OF THE DISCLOSURE

Herein disclosed are exemplary embodiments of a bio-recognition sensor comprising: a polymer scaffold which comprises an electrode, and an electropolymer; wherein the electropolymer forms a coating on the electrode; and a template, wherein the template comprises: a three dimensional imprint of a biological structure in the electropolymer; wherein the template comprises chemical moieties that are selective for binding to the biological structure. In some embodiments of the bio-recognition sensor herein described the electrode comprises carbon nanotubes. In another embodiment of the bio-recognition sensor herein described, the electropolymer comprises polyphenol, in a further embodiment the coating is about 5 nm to about 25 nm thick; in another embodiment the coating is about 10 to about 20 nm thick, and in a further embodiment the coating is about 15 nm in thickness.

In an embodiment of the bio-recognition sensor described herein the chemical moieties are comprised of oxidation species of the electropolymer, in another embodiment the oxidation species comprise ortho, meta and para phenolic monomers, dimers and trimers.

An further embodiment herein describes a method of making a bio-recognition sensor comprising: (a) forming a electropolymer coating on an electrode; (b) binding a target biological structure to the coating to form an imprint; (c) removing the biological structure from the coating; and (d) forming a template; wherein the template binds a specific biological structure. In another embodiment the method of making a bio-recognition sensor further comprises adding pre-complexation moieties after step (a) and prior to step (b), wherein the pre-complexation moieties bind to the biological structure of step (b).

In another embodiment, a method of making a bio-recognition sensor comprises (a) forming a electropolymer coating on an electrode; adding pre-complexation chemical moieties; adding a target biological structure; binding the pre-complexation chemical moiety to the target biological structure; binding the target biological structure comprising the pre-complexation chemical moiety to the coating to form an imprint; removing the biological structure from the coating; retaining the pre-complexation chemical moiety bound to the electropolymer coating; and forming a template; wherein the template comprises specific chemical binding moieties selective for the biological structure.

In some embodiments of the methods of making a biosensor herein described, the electrode is comprised of carbon nanotubes, in another embodiment of the methods of making a biosensor the electropolymer comprises polyphenol, in a further embodiment of the methods of making a biosensor the coating is about 15 nm in thickness. In another embodiment of the methods herein described, the pre-complexation chemical moieties comprise chemical compounds with large free energy of binding for the biological structure. In some embodiments herein described, the pre-complexation chemical moieties are identified by: docking a library of small molecules to the biological structure in silico; identifying compound clusters, wherein the compound clusters are docked at a specific binding motif, calculating a free energy of binding of a representative compound of each the cluster; wherein the compound with the greatest change in free energy of binding to the biological structure is selected as a pre-complexation chemical moiety.

In some embodiments of the methods of making a biosensor herein described, the electrode is comprised of carbon nanotubes. In another embodiment, the electropolymer comprises polyphenol. In a further embodiment of the methods of making a biosensor, the coating is about 15 nm in thickness. In another embodiment, the electropolymer is a nonconductive polymer, and in a further embodiment the non conductive polymer comprises 3-aminophenol, 3-methyphenol, 3-nitrophenol, 1,3-dihydoxybenzene, 1,2-dihydroxybenzene, 1,4-dihydroxybenzene or a combination thereof.

In another embodiment of the methods herein described, the pre-complexation chemical moieties comprise chemical compounds with large free energy of binding for the biological structure. In some embodiments herein described, the pre-complexation chemical moieties are identified by: docking a library of small molecules to the biological structure in silico; identifying compound clusters, wherein the compound clusters are docked at a specific binding motif, calculating a free energy of binding of a representative compound of each the cluster; wherein the compounds with the greatest change in free energy of binding to the biological structure are scored as candidates. In some embodiments, a molecular dynamics will run to simulate the interaction between each candidate and the biological structure to verify the characteristic binding motif; in other embodiments, the molecular dynamics will run a competition of the candidates to bind with the biological structure, so that a final score of the candidates will be obtained to design the imprint with designated affinity and binding sites.

In an embodiment herein described, the biological structure is a native protein, a protein comprising a mutation, a protein comprising a post-translational modification, a molecule; or peptide. In another embodiment, a method of detecting a biological structure herein described comprises adding a sample comprising a biological structure to a bio-recognition sensor; wherein the sensor comprises: polymer scaffold comprising an electrode, and an electropolymer, wherein the electropolymer forms a coating on the electrode; and a template, wherein the template comprises: a three dimensional imprint of a biological structure in the electropolymer; wherein the template comprises chemical moieties that are selective for binding to the biological structure; and measuring a change in current leakage by the electrode, wherein the change in leakage is indicative of binding of a biological structure with selectivity for the template. In a further embodiment of the method of detecting a biological structure, the biological structure is a biomarker indicative of a disease, in a still further embodiment the sample comprises a biological sample from a subject in need thereof of detection of a biomarker for a disease state, grade, or subclass. In another embodiment, detection of said biomarker for said disease aids in selection of a therapeutic for a disease treatment, and in a further embodiment the sample comprises a biological sample from a subject in need thereof of detection of a biomarker for the prognosis of a disease.

In some embodiments, a method of detecting a biological structure is herein described, wherein said method comprises adding a sample comprising a biological structure to a bio-recognition sensor; wherein said sensor comprises a polymer scaffold comprising an electrode and an electropolymer, wherein said electropolymer forms a coating on said electrode; and a group of imprints, wherein said imprints comprise three dimensional molds corresponding to each of a group of biological structures in said electropolymer; wherein said imprint comprises chemical moieties that are selective for binding to said biological structure; and measuring a change in current leakage by the electrode, wherein a change in leakage is indicative of binding of a biological structure with selectivity for said template. In some embodiments the biological structure is a group of biological structures. In some further embodiments the biological structure is a group of biomarkers indicative of a disease. In one embodiment of the method of the pre-complexation chemical moieties are identified by: docking a library of small molecules to said biological structure in silico; identifying compound clusters, wherein said compound clusters are docked at a specific binding motif, calculating a free energy of binding of a representative compound of each said cluster; wherein said compound with the greatest change in free energy of binding to said biological structure is selected as a pre-complexation chemical moiety, and verifying the binding characteristics by molecular dynamics that extracts the binding interfaces and scores the competition of the chemical moieties.

In some embodiments, the biological structure is from a group of molecules that belong to a proteomic profile. In other embodiments the profile comprises a group of biological molecules identified as a biomarker for a disease state, grade, or subclass, in further embodiments, detection of said biomarker results in selection of a therapeutic for a disease treatment, and in further still embodiments detection of said biomarker results in a prognosis of a disease.

### BRIEF DESCRIPTION OF THE DRAWINGS

FIG. 1 (Scheme 1): depicts an embodiment of an oligomer-template assembly in an electropolymerized MI to render specific recognition, in one embodiment the electropolymerization on the electrode (black) produces various oligomers. The template molecules in the solution selectively binds with some of the oligomer, “favored” oligomers are deposited with the “unfavored” oligomers on the electrode, and form recognition features in the MIs, as described in herein;

FIG. 2A-B: depicts an embodiment of an ultra-sensitive protein sensing (bio-recognition) with EIS (Electrochemistry Impedance Spectroscopy) and DPV by a CNT nanosensor. The sensor was imprinted with the hFtn. Some embodiments of the relationships between the EIS and DPV responses to protein bindings are presented. FIG. 2A: for the hFtn, its concentrations range from 10⁻¹² to 10⁻⁷ g/L. In some embodiments no impedance change was observed at 10⁻¹² g/L. FIG. 2B: for the non-specific bindings of bovine serum albumin, horse ferritin and horse apoferritin, the protein concentrations are up to about 10⁻⁴ g/L;

FIG. 3(A-C) depict embodiments of the electropolymerization of PPn on CNT electrodes and protein entrapment; (A) depicts the voltammogram of self-limiting PPn electropolymerization on the CNT array; (B) depicts the PPn coating and the entrapped hFtn molecules. The iron(ic) crystalline core of hFtn offered contrast under transmission electron microscope; and (C) depicts the EDX spectrum obtained in a TEM field with the CNT subject to PPn electropolymerization and the hFtn entrapment. As the only source of iron was the hFtn molecules carrying ironic core, the Fe peaks confirmed the presence of hFtn;

FIG. 4A depicts an electropolymerization and electrochemical recording system, wherein the electropolymerization was conducted by cyclic voltammetry with the working electrode (W) potential ramping between 0 and 900 mV vs. The reference electrode (R). Pt wire was used as the counter electrode (C). A self-limiting reaction was indicated by the Faradic current that drastically dropped after the first cycle. The Reference 600 system was in charge of both electrochemical deposition and electrochemical recording. FIG. 4B shows the principle of protein detection with the imprinted CNT nanosensor. The PPn coating is deposited at the tip of a CNT. The imprints introduce leakage current and decrease the CNT electrode impedance, and the target protein binding to the imprints will “plug-in” the imprint vial in the coating and increase the sensor impedance Z(w). FIG. 4C depicts the recording of protein binding with the sensor. Electrochemistry Impedance Spectroscopy detected the sensor signals in the sequential status of the sensor: as-prepared CNT chip (no PPn)→with PPn coating→after hFtn MI developing (hFtn imprinted coating)→hFtn rebinding (10⁻⁷ g/L hFtn). Differential Pulse Voltammetry (DPV) also showed signals corresponding to MI development (no hFtn) and rebinding to targets (10⁻⁷ g/L).

FIG. 5A and FIG. 5B depict the docking of phenolic compounds on proteins: FIG. 5A depicts the docking of Compound 12 on E7 protein in the strongest field (binding energy of ΔG=−6.73 kJ/mol). Its location is presented in a whole protein and a close-up view. The surface atoms of carbon, nitrogen, oxygen, and sulfur are displayed in gray, blue, red, and yellow. The green circles, diamonds, and triangles indicate the scope of atom displacement within the cluster for each protein. FIG. 5B depicts the calculated docking clusters on proteins: the axes show levels of binding force field. The size of the symbols is proportional to the cluster size. The phenol cluster size is presented with a scale-down by a factor of 0.5. On each protein, the density of the circle, diamonds and triangles that represent each compound is considered the amount in a cluster. The numbers (1-14) in the top panel depicts the range of ΔG from the docking of the eleven regular fMer (wherein fMer is a functional monomer) molecules on E7 protein. For each compound, the data collected is the result of 100 docking procedures (runs) with the template protein in accordance with embodiments herein presented, the ΔG from the docking of the p-fmers (compounds 1-14) can be seen to be more negative, i.e. exhibiting tighter binding; The full chemical names of compounds described herein are listed in Table 2.

FIG. 6: depicts interfaces formed by phenolic compounds on the proteins. (A) Phenolic Compound 1 (left) and Compound 12 (right) with different distributions of docking sites. (B) Docking zones of phenolic compounds on the protein front and backsides. Each dot (red) represents a docking site, i.e. a cluster. The docking zone on each protein encompasses the calculated clusters of all fourteen compounds in accordance with embodiments herein presented;

FIG. 7(A-B): depicts an embodiment of protein detection with tuned imprint affinity as described herein: (A) depicts a scheme showing the customized interface in the three dimensional imprint surface. The upper row shows the E7 protein and Compound 12 used as the template and p-fMer respectively. After their pre-complexation, the p-fMer pre-occupies its binding sites (red-site). The electropolymerized PPn fills in the unoccupied areas (gold) later to complete the interface; the binding site represented by the interface maintains the same shape as that when electropolymerized without p-fMer addition, while its affinity with the target is modified. The comparison of the protein detection with sensors fabricated following the two approaches is shown in FIG. 7(B). The CaM was used as the template and correspondingly the Compound 2 was the p-fMer. The “A” values represent the relative increase in modulus of impedance (Z_{mod}^{Ca}) due to Ca-CaM rebinding at given [Ca²⁺] comparing to the impedance at [Ca²⁺]-free)(Z_{mod}⁰). The data are fitted with the Hill equation in accordance with embodiments herein presented.

FIG. 8 (A-B). depicts a further embodiment of hypothetical protein recognition in the electropolymerized MI on the nanosensor following the method of Cai, D. et al. Nature Nanotechnology (2010) 5, 597-601, incorporated herein in its entirety by reference); (A) depicts that oligomers (shown in different shapes) are produced on the CNT nanoelectrode (black). Some of the products (yellow) have higher affinity with the template protein (blue) than others (red), and complex with the protein. The oligomers complexed with the template (pfMers) render the binding interface in the imprint structure; and (B) depicts heterogenetic structures of the polymer product in the electropolymerization.

FIG. 9 (A-D) again depicts an embodiment of the oligomer concentration during electropolymerization of PPn in an embodiment herein described; (A) [shows self-limiting faradic current in five voltammetric electropolymeirzation cycles; (B) shows PPn coating on a nanoelectrode array; the top view with a scanning electronic microscopy. The array of nanoelectrodes (red) follows the pattern masked with a microbead monolayer (yellow). The dashed parallelogram contains two tips. It is used as the unit area to estimate the total tips on the chip, the bottom shows the transmission electron microscopy of a single CNT tip coated with electropolymerized PPn; (C) shows a comparison of PPn deposition volumes estimated with two methods; (D) shows the oligomer concentration profiles calculated according to the diffusion of dimer molecules produced at each nanoelectrode in the first voltammetric cycle. The traces are: time courses of the faradic current (red), and dimer concentration correspondingly at 20, 40 and 100 □m from the electrode. The three 3D profiles are the dimer concentration profiles in a 400×400 mm² area around a nanoelectrode at 5, 10 and 15 s after starting the voltage cycle.

FIG. 10(A-C) depicts the contact between pfMer and E7 protein of an embodiment herein described(A) Depicts an overlay of OLS trajectory on E7 sampled every 200 ps from the 100 ns-simulation (Molecular dynamics simulation). OLS is in ball-and-stick. The OLS molecules not in contact with the E7 are excluded. E7 backbone is illustrated in ribbon. Its surface is also outlined in silver. In (B): a contact is counted when a pfMer atom is less than 4 Angstroms (dashed lines) to a residue atom (Val:C). The total number of contacts is obtained after all pfMer atoms are taken into account. Color representation: Carbon (cyan), Oxygen (red), Nitrogen (blue), Hydrogen (gray). (C): shows an evaluation of contacts with E7 residue. (top) Contact distributed in residues. The contact number represents contacts averaged over total frames. Standard deviation is shown as bar. (Bottom) Score of contact in residues. A contact score was defined as: Contact score=Contact number/Deviation. Dashed line marked score threshold 1.2, an arbitrary criterion to identify frequent contact residues (red). E7 residue ID from 1 to 56 represents amino acid sequence:

(SEQ. 1)
GSHMAEPQRHKILCVCCKCDGRIELTVESSAEDLRTLQQLFLSTLSFV
CPWCATNQ;.

FIG. 11(A-C): depicts an embodiment of the method of ultrasensitive protein sensing with EIS and DPV by a CNT nanosensor as described herein. The sensor was imprinted with the human ferritin (hFn); (A) depicts electrochemical impedance spectroscopy (EIS) with the nanosensor under conditions: as original (no PPn), after PPn coating (PPn coating), after imprint development (hFn imprinted PPn), after imprint rebinding with hFn at 10⁻⁷ g/L (10⁻⁷ g/L hFn); (B) depicts differential pulse voltammetry (DPV) recording of leakage current in the nanosensor: after imprint development (no hFn), and after imprint rebinding with hFn at 10⁻⁷ g/L (10⁻⁷ g/L); and (C): shows that the signals from EIS and DPV recording are correlated with the hFn rebinding when the concentration increasing from 10⁻¹² to 10⁻⁷ g/L, while no concentration dependent correlation is shown in the non-specific binding of bovine serum albumin.

FIG. 12: depicts the contact energy between OLS pfMers and E7 residues. The electrostatic energy (eStatic) and van der Waals energy (VDW) are overlaid for comparison. The highly scored residues in FIG. 10, (C) are highlighted in red.

FIG. 13: depicts the contact interfaces of pfMers with E7 protein of an embodiment of the invention herein described. Based on the contact score, the frequent contact residues with OLS, OSE and OET are marked with solid a block in the sequence. Three shared contact regions, i.e. R1, R2 and R3, are in boxes which are further highlighted by an additional box. Each of them form a continuous surface in the protein. Their residue positions are visualized in the protein (ribbon) structure with different kinds of color: R1, yellow (corresponds to first boxed region, 25-30AA, indicated by the first arrow); R2, green (corresponds to third boxed region, 45-50AA, indicated by the third arrow); R3, light blue in the front, purple in the back (corresponds to the second highlighted box, 37AA-52AA). Opposite sides of E7 protein are also shown.

FIG. 14(A-B): depicts the contact of OSE-E7 (A) and OET-E7 (B). Contact values represent contact number per frame. Highly frequent contacts (red) are identified with score 1.2.

FIG. 15 (A-C): depicts an embodiment of the method herein described, wherein the competition of pfMers with the E7 protein. (A) shows RMSF values of the E7 residues in the four follow-up competitions between OLS and OET. (B) is a summary of pfMer contacts with E7 residue from the competition. Error bar stands for standard deviation. (C) Shows the time course of pfMer contacts in the competition for R2: (45,46,47,48). The data is from an embodiment of a 20 ns follow-up simulations. The number in each panel is the ID of residues.

FIG. 16: depicts the contact map of OLS60 and OET63, which closely interacted in the simulation for 100 ns. R1, R2 and R3 indicate the contact region extracted based on contact scores and outlined in FIG. 13. A highly dynamic scenario at the first 5 ns is magnified. Inset: the relative position of the pfMers and the E7 protein at 0.5, 1.5, 2.5 and 4.5 ns.

### DETAILED DESCRIPTION OF THE DISCLOSED EMBODIMENTS

In some embodiments, a method is disclosed to synthesize polymeric materials containing specific binding sites for a target molecule (such as a biomarker). In some embodiments of MIs described herein, template molecules are entrapped in the crosslinking polymer and extracted later to create three-dimensional cavities within the polymeric matrix.

In some embodiments, the template will pre-complex with functional monomers ((fMers), for example compounds or amino acid residues that show a preferential binding for positions on the template structure, such as to mimic the binding moieties of a substrate) by binding with particular residues on the template. In some embodiments the fMer will stay in the cavity imprint, so that the cavity will be complementary to the template (or target at the stage of rebinding) not only in the size and shape, but also complementary in functional groups.

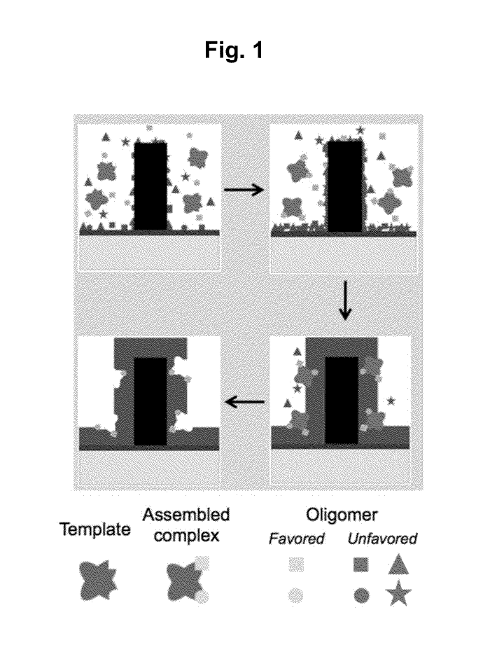

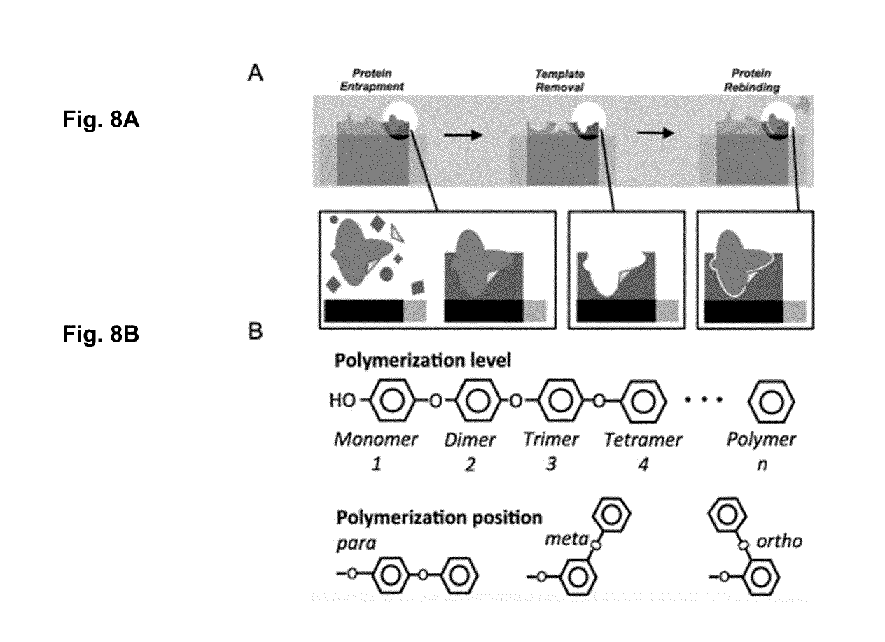

The electropolymerized polymers described herein are therefore not simply morphologically matched to the templates within the polymer network²¹, herein the protein (template) imprinted on the CNT electrode was fabricated by the electropolymerization mechanism as seen in FIG. 1 (Scheme1, or see similar depiction in FIG. 8(A)) which depicts an embodiment of the molecular imprinting principle described herein, wherein in one such embodiment, CNT-electropolymerized oligomers with random structures can either deposit on the electrode surface derived of carbon nanotubes (CNT) shown in black, or diffuse into the surrounding solution. The later interact with the protein templates in the solution and assemble into complexes (i.e. certain molecules or oligomers with certain chemistries and conformations will favorably bind to the protein template forming complexes). The complexes with energy favorable binding sites are finally deposited on the CNT electrodes. Those oligomers included in the template and occupying the substrate binding sites will be fixed in the MI as recognition sites after the extraction of the protein component of the template. In some embodiments, in silico oligomer docking on the protein surface identified specific targeting areas, and herein where an oligomer was introduced to the assembly at the specific target areas an improved MI-target binding affinity and increase in sensitivity of the sensor was observed.

Embodiments of the method of electro-polymerization of the coated electrode, imprinting of the coating to form a template, (wherein the template may comprise pre-complexation moieties either formed by oxidation species of the electrode, or by addition of specific small molecules that were found to comprise very large negative free energies of binding (from docking studies as described herein)), removal of the protein template by washing, and measuring of subsequence specific binding of a target protein by current impedance and leakage measurements are exemplified herein.

In some embodiments, imprint fabrication for protein detection requires the following steps: (1) protein template entrapment in the electro-polymerized polyphenol coating on the sensor electrode; (2) removal of the entrapped template from the polyphenol coating to form the 3D vial, i.e. imprint, of the template protein; and (3) selective rebinding of the target protein (the same protein molecule as the template) to the imprint by bio-recognition to generate the detection signals. Since the polyphenol is non-conducting polymer, the binding signal can be detected by the measurement of either the impedance or the leakage current in the sensor. The two approaches are correlated as following: when the imprint is fabricated a structural defect in the polyphenol coating is produced leading to the drop in impedance and increase in leakage current; when rebinding happens between the imprint and target, it is equivalent to fixing the defect of the coating, whereby the impedance can be recovered and the leakage is blocked.

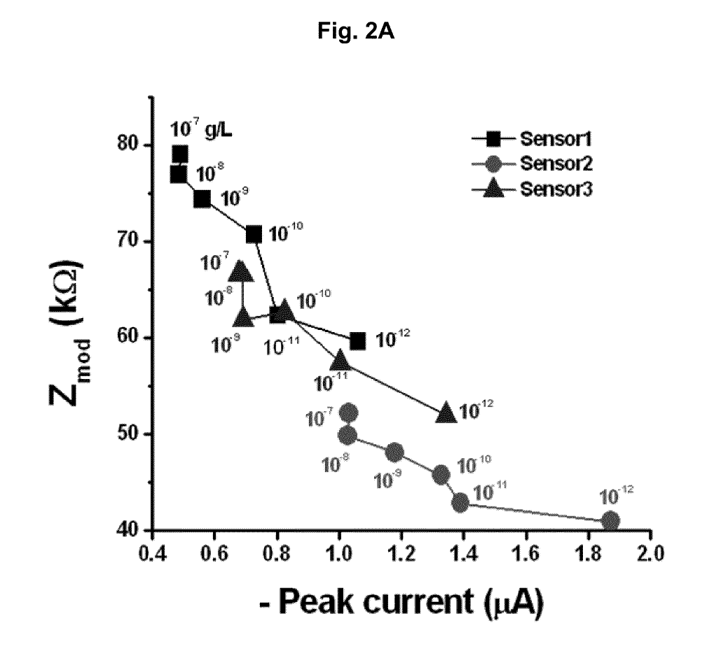

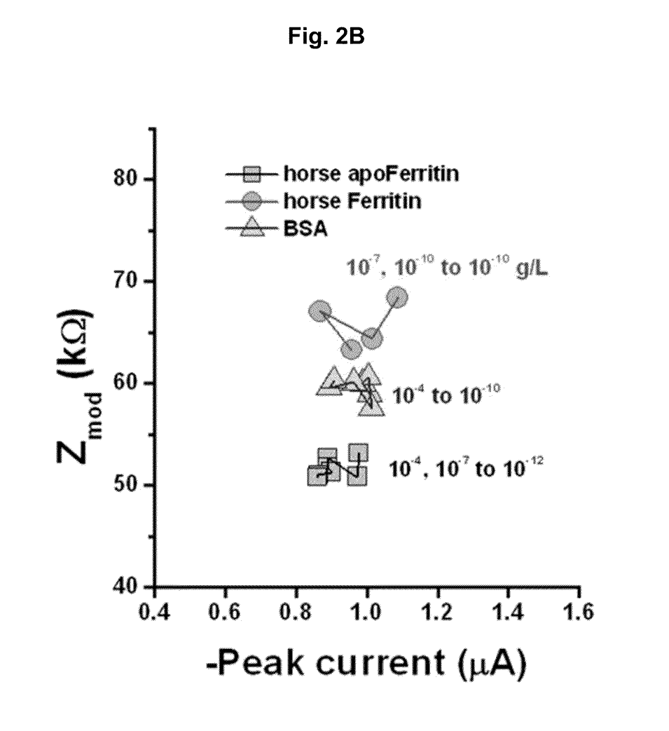

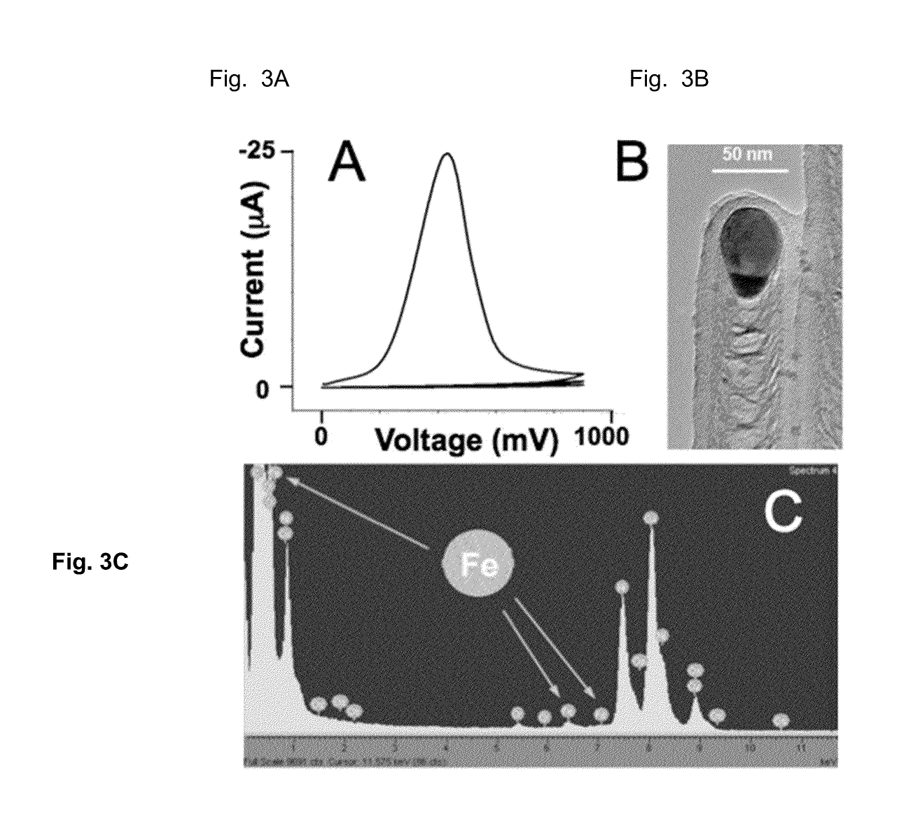

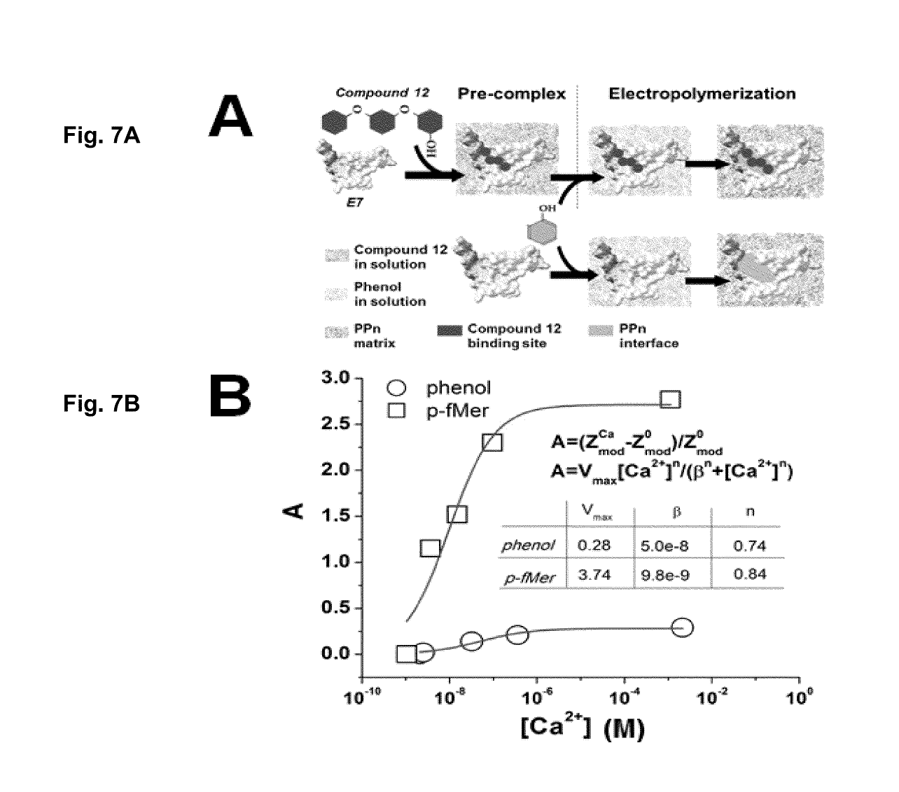

Protein detection as described in FIG. 7(A-B) was conducted by impedance and leakage current measurement (FIG. 2). The results of the two methods described above, were well correlated and reflect the selective and sensitive responses mediated by the MI. In fact, the electro-polymerization of polyphenol (PPn) nanofilm on the CNT array may be of key to sensor platform design. The phenol oxidation is a self-limiting process because of the drastic ohmic potential drop across the PPn film by the non-conductive PPn. Typically, a large Faradic current peak only appears in the course of the first cyclic voltammetry (CV) scanning. Under transmission electron microscope (TEM), the coating thickness on CNT is 15±3 nm (n=20) and is comparable to the diameter of human ferritin (hFtn) molecules (FIG. 3). It is also noticeable that the coating is thinner than that deposited on a planar metallized electrodes, and may be due to two reasons: first, the decaying electric field along the radial direction of the nanotube limits the spatial distribution of the oxidation, as addressed by the Cottrell equation, the reactions on “ultramicroelectrode” is more intense than that on a planar electrode, since the oxidation rate is not limited by the depletion of electroactive species;²² and it can also be reflected in the PPn deposition which based on the Faradic charge Q_{CNT}, the volume of PPn (V_{PPn}^{Cal}) can be estimated by:

$${V}_{PPn}^{Cal} = \frac{m \cdot {Q}_{CNT}}{F \cdot σ} \qquad ( 1 )$$

where m is the molecular mass of phenol, F is the Faradic constant and σ is the density of phenol and equals to 1 mg/mm³.

Alternatively, the volume of PPn (V_{PPn}) on the CNT can be calculated according to the actual thickness of PPn coating, the total number of CNT on chip and the dimensions of CNT.

Both values are demonstrated in FIG. 3. In some embodiments it is noticed that:

V_{PPn}^{Cal}=kV_{PPn} (2)

k≈10±2), wherein only about 10% of the amount of oxidized phenol molecules were deposited on the CNTs. Most, otherwise diffused as oligomers in the solution, or stopped as side products.²³ In contrast, the k value between 1.16 and 1.21 was measured with a quartz crystal microbalance (QCM) for the PPn deposition on a planar gold electrode.²⁴ It indicates that the CNT mediated electro-polymerization can produce excessive amount of phenolic radicals and small oligomers near the CNTs. Such a system is required to carry out the interaction between the template and the PPn oligomers, as illustrated in FIG. 1 (and similarly in FIG. 8(A)). In such an embodiment of an assembly mixture, heteropolymers from ortho-, meta- and para-dialkoxy benzene units will be polymerized in various degrees.^{25,26} The diversified small PPn products will assemble with the protein (target) molecules following their energy preferences. Compared to the traditional imprinting protocols employing the fMer-complexation pre-treatment, the CNT mediated electro-polymerization can provide the assembly system that hosts a group of “fMer” to simultaneously interact with the template. It is noted herein that the term “about” defines a numerical range of +10% or −10% of a given value, wherein for example “about 80 units” encompasses +8 units or −8 units, i.e. a range of 72 units to 88 units.

The in silico simulation of oligomer docking with AutoDock4 on the target protein molecules were in one embodiment used to determine: (1) the binding energy between the small polymer of different building blocks and the template protein molecule, and (2) the specific polymer binding sites on the protein. Several small proteins, i.e. human papillomavirus E6 (18 kDa) and E7 (17.5 kDa) protein, and bovine testicular CaM (16.7 kDa), were used instead of the large hFtn (450 kDa). Fourteen regular phenolic compounds were analyzed, in parallel to 11 fMer molecules.

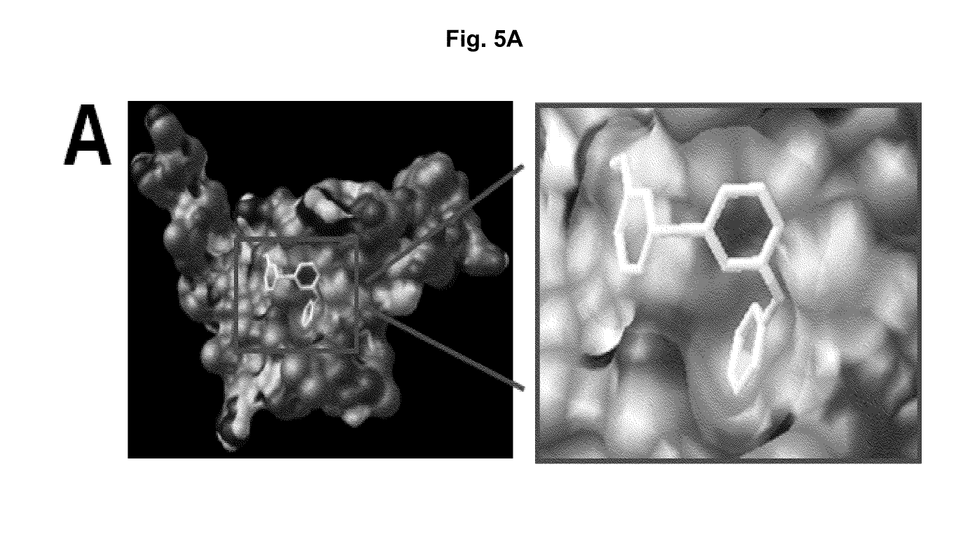

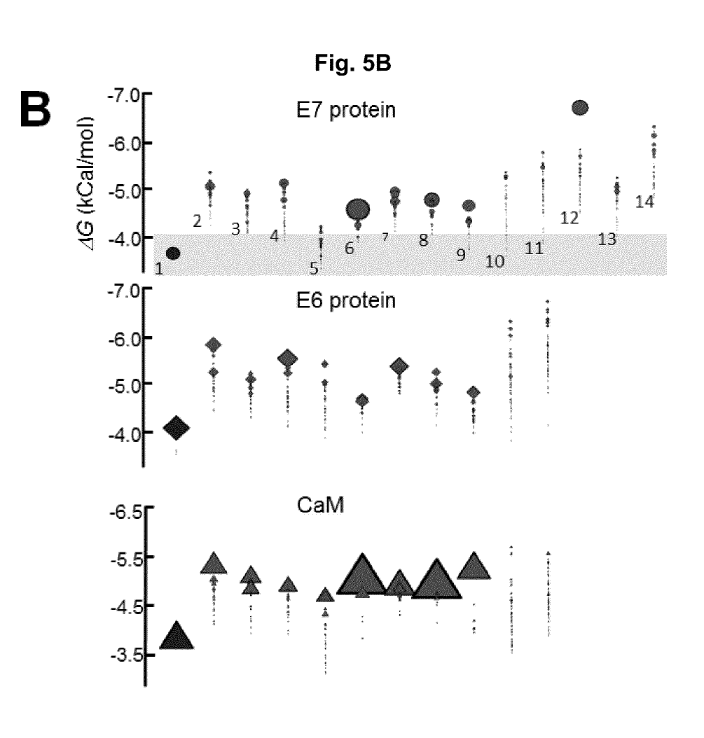

As described above, the force field ΔG, i.e. the free energy change corresponding to the molecular interactions, integrates the energy evaluations of molecules, as well as the conformational entropy lost upon binding. The free energy calculation includes the van der Waals force, hydrogen bonding, electrostatics, and desolvation. The ΔG indicates the binding energy, binding force and affinity between the protein and polymeric compounds. Among 100 runs of docking simulations per compound per protein, the largest ΔG values are summarized in Table 2. In some embodiments the electropolymerized phenolic molecules have higher binding energy (ΔG) to the proteins than the conventional fMer molecules do. For example, the largest ΔG of E7-Compound ranges from 4.22 to 6.73 kcal/mol versus that for E7-fMer from 2.05 to 4.0 kcal/mol. The corresponding docking of E7-Compound12 is shown in FIG. 5A. The compound's benzene end occupies the hydrophobic pocket at the center of E7 surface. Its hydroxyl group falls in a stable interaction with polar residues on the protein.

A cluster is formed if multiple dockings end at the same location (with displacement between each less than 3.5 Å) on the protein. A cluster suggests a potential binding site with the protein. However, as shown in Table 1, the amount of clusters, cluster size (i.e. number of dockings that fall in the same cluster), and ΔG values of the Compound 10 and 6 dockings on the CaM protein are very different. The CaM-compound 6 exhibits preferential bindings. About 95% of the docked structures produced two clusters. In contrast, compound 11 has no specific preference of docking. Among a total of 87 clusters, the biggest sized cluster had only four molecules docked, indicating a broad distribution and poor specificity of bindings on the CaM. The simulation results are summarized in FIG. 5B, and collectively demonstrates the ΔG and the size of each cluster on each of the three proteins. FIG. 5 also shows the energy preferential, and selective complexations between the oligomers and proteins, which in some embodiments may be analogus to the template-fMer pre-complexation in conventional imprinting procedures. Phenolic fMers (p-fMers) therefore may be computationally identified from the PPn compounds. For example, the trimer Compound 12, and dimer Compounds 6, 8, 9, may be p-fMer candidates for E7; dimer compounds 2, 4, 7 for E6; and dimer compounds 2, 6, 8, 9 for CaM. The protein-p-fMer pairs all exhibit a small number of clusters, comprise a big cluster size (members of the cluster), and high binding force. Pre-complexation of such p-fMers with the template before the electropolymerization may convey an artificial manipulation of the MI-target affinity.

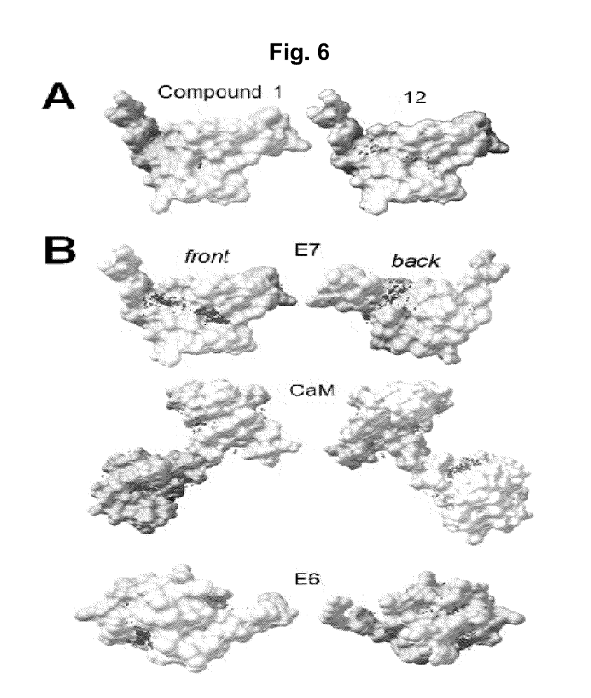

Another docking characteristic identified herein are the spatial distribution of the docking sites. In FIG. 6A, cluster locations of phenolic compounds 1 and 12 on E7 protein are compared. The docking of phenol monomer (compound 1) is mostly entrapped in the small hydrophobic pockets on the protein. Structural complexity in compound 12 makes their binding to the protein more distributive than the phenol monomer. There are in total 42 clusters with size ranging from 29 structures to 1 for the docking of E7-compound 12. In FIG. 6B, all clusters are overlaid. It gives an overview of all oligomer dockings that simultaneously happen during the course of electro-polymerization. In some embodiments, common binding zones emerge on E7, CaM and E6 proteins. This is an implication of the imprint structure, because such ensembles may be fixed in the polyphenol coating, they then serve as binding features in the protein imprints.

Further, The protein interface incorporates the spatial orientation of chemical groups, surface topological features, and force fields of the surface residues of amino acids. A similarity can be noticed between the natural bio-recognition and the scenarios shown in FIG. 6. That said the hetero-p-fMers derived from a common phenol monomer by electro-polymerization may have similarities in their binding properties and assemble as an interface to the template protein, therefore in some embodiments molecular dynamics may be used to show the pre-complexation process and the heterogeneity of molecular imprint recognition sites as reported before, as detailed below. In some embodiments, the dynamic information may provide information to the cooperative interactions during the interface formation and polymer renaturation processes, and guide the optimization of the electro-polymerized protein imprints.

As the imprint interface is a mix of p-fMers that have a broad variation of binding forces, alteration of concentrations of certain contents may in some embodiments bias the affinity of the imprint while maintain the original overall morphology of the interface. The principle of MI manipulation and optimization is illustrated in FIG. 7A. To prove that, a CaM imprint sensor was modified by artificially increasing the amount of p-fMer Compound 6 (the largest delta G=−5.02 kJ/mol) (FIG. 7B). The CaM saturated with Ca²⁺ was used as the template, so that the imprint kept the conformation of calcium bound CaM (Ca-CaM). The CaM sensing samples were prepared in buffers with various levels of free Ca²⁺ to determine different concentrations of Ca-CaM exposed to the sensor. The free Ca²⁺ concentrations were buffed with a Ca²⁺ chelator, ethylene glycol tetraacetic acid (EGTA). The A value denotes the relative increase of sensor impedance corresponding to target binding. It was largely increased by the pre-complexation with the compound 6 (marked as p-fMer). In the Hill fitting of the sensor responses, the p-fMer sensor has the Vₘₐₓ value about 10 times larger than the phenol sensor implicating a tighter binding between the imprint and protein. The B value denotes the dissociate constant between the protein and the imprint. The p-fMer imprint (B=9.8 nM) has higher affinity than the phenol one (B=50 nM).

### EXAMPLES

Based on the principles described above, disclosed herein in some embodiments are the formation of a high quality protein imprint for reducing the LOD of biomarker sensors. In some embodiments, electropolymerization of PPn on the CNT array includes the ensemble of the instantly produced heteropolymers with the template protein. The in silico simulation indicate the formation of a synthetic interface in the electropolymerized protein imprint. MI affinity can be improved by pre-complexation with computationally screened p-fMers to meet the requirements of early detections of diseases.

### Reagents and Instrumentation:

Phenol, phenol oligomers, CaM, and ferrocene carboxyl acid (FCA) and bovine serum albumin were obtained from Sigma-Aldrich (St. Louis, Mo.). Human ferritin protein was obtained from AbD Serotec (Raleigh, N.C.). Horse ferritin and apoferritin were obtained from MP Biomedicals (Solon, Ohio). Phosphate buffered saline was obtained from Fisher Scientific (Pittsburgh, Pa.). Electropolymerization and electrochemical behavior of thin-films were conducted with a Reference 600 electrochemical system supplied by Gamry Inc. (Warminster, Pa.) operating via Framework. Data analysis was conducted with Echem Analyst. TEM images of PPn coated CNTs were obtained with a JEM-2010F TEM (JEOL, Tokyo, Japan) operating at 200 kV.

### Development of an Electrode [CNT Array Preparation]:

In some embodiments array fabrication begins with CNT growth via plasma-enhanced chemical vapor deposition on polystyrene sphere-patterned substrates, followed by embedding in SU8-2002 photoresist polymer, and then mechanically polished to expose the tips. First, SU8 was spun on an array at 3000 rpm for 30 s. [13] Following a soft bake for 5 min at 100° C., SU8 was cross-linked by exposure to UV light for 3 min and then the sample was incubated at 150° C. overnight. Lastly, the chip was polished with a vibratory polisher (Buehler in Lake Bluff, Ill.) with 80% power level for 6-9 hrs until the pattern was revealed (FIG. 3A).

### Electrochemistry Experiments:

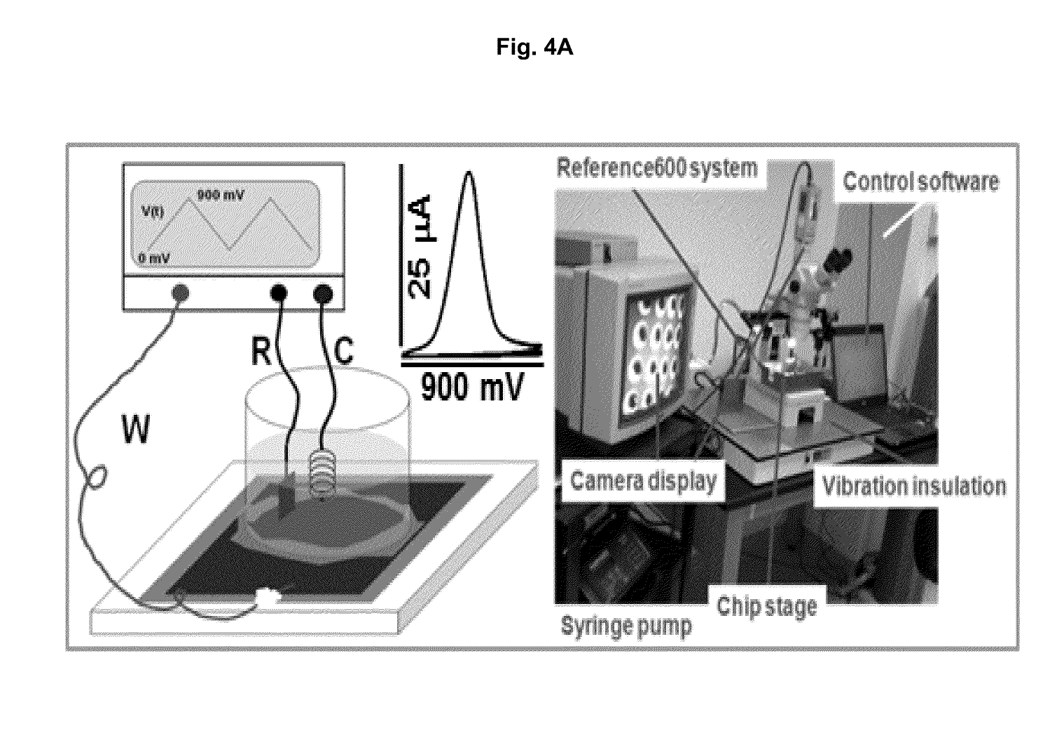

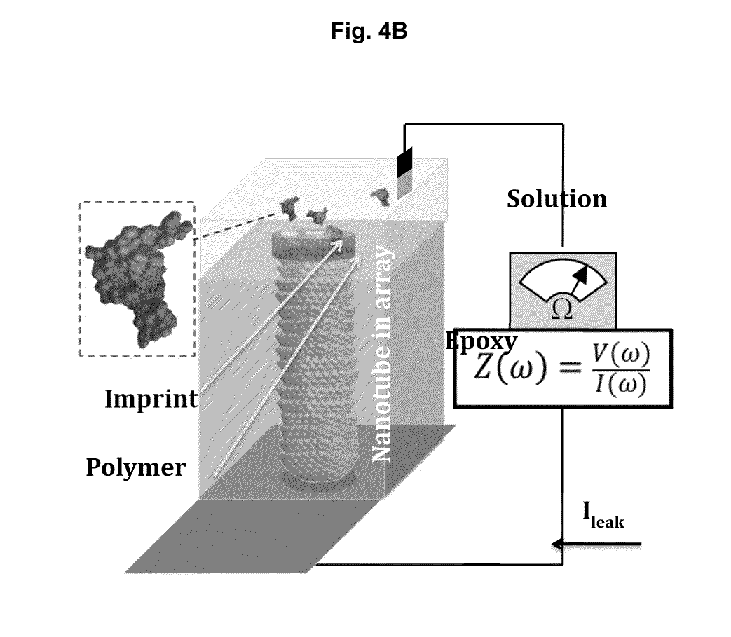

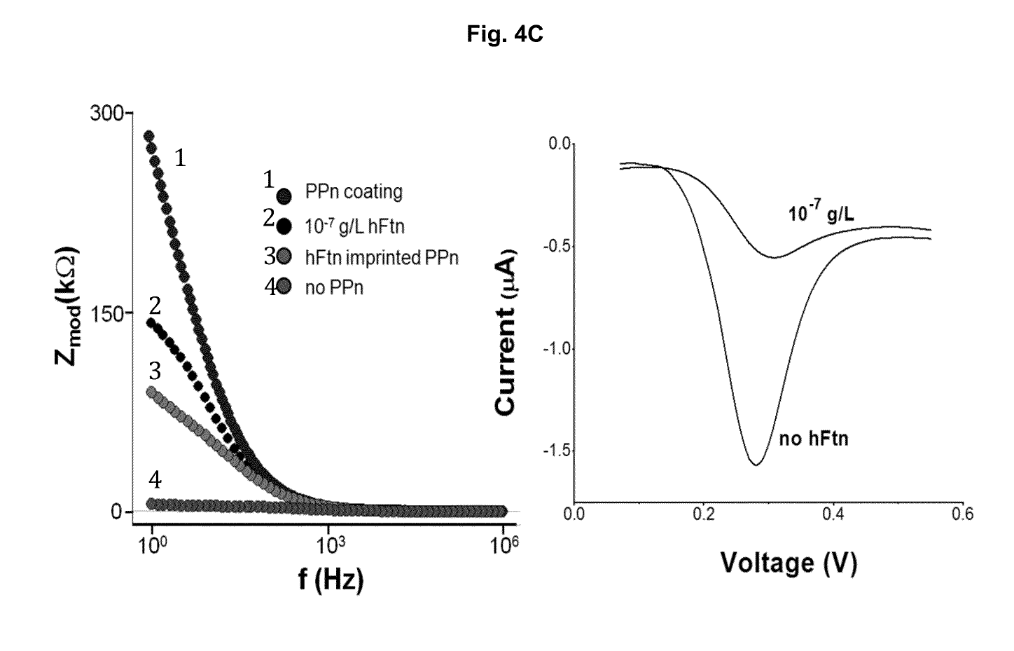

As described above and illustrated in FIG. 4(A-C), three-electrode electrochemical systems were formed by connecting the CNT chip as the working electrode and using chlorinated silver and platinum wires as reference and counter electrodes, respectively. PPn film was deposited on the CNT array by cyclic voltammetry in a phenol (1.5 mM) supplemented phosphate buffered saline (PBS)(deposition buffer), pH=7.4. The potential on the working electrode was scanned 5 times between 0 to 0.9 V vs. the reference electrode. In order to entrap ferritin in the PPn coating, the protein (100 pg/ml) was added to the PPn deposition buffer. Following electrophoretic attraction by applying 300 mV DC voltage for 30 s, cyclic voltammetric voltages were used to form the PPn coating as described above for co-deposition of ferritin. A similar procedure was used to entrap CaM in the PPn coating with the exception that during CaM co-deposition, 1 mM Ca²⁺ was included in order to render a conformation with a full scale elongation (“open”) that offered distinctive imprint morphology from its globular shape at Ca²⁺ free or other partial “close” status. EIS was conducted before and after the PPn deposition to evaluate the impedance properties of the electrode surface and its interface to the buffer solution containing FCA (1 mM) in PBS. The sine wave was 10 mV peak-to-peak in amplitude. It was superimposed on a 300 mV DC voltage. Frequency was scanned from 1 Hz to 1 MHz. The impedance data were fitted to an electrical equivalent circuit using the impedance analysis function in Echem Analyst software. DPV was conducted in the same buffer as that for EIS. Initial and final potentials vs. reference electrode were 0 and 0.5 V respectively. Pulse size was 50 mV and pulse time was 0.05 s; step size was 2 mV, and sample period was 0.1 s.

### Protein Imprint Development:

For imprint development, the sensor with ferritin-entrapped PPn coating was rinsed and incubated overnight in deionized water at room temperature. Alternatively, a developing buffer containing acetic acid (5% w/v) and sodium dodecyl sulfate (SDS) (10% w/v) was used for higher protein extract efficiency. After ferritin entrapment and removal, the sample was evaluated by TEM and EIS (FIG. 3B and FIG. 4C). To prepare TEM samples, coated CNTs were carefully scraped off with a sharp blade in isopropanol. About 10 μl of CNT suspension was dropped onto TEM copper grids with carbon film. The samples were checked with TEM immediately after isopropanol was vaporized. With this specimen preparation, the cross-section of the coated CNTs could be observed by TEM bright field imaging, as shown in FIG. 3B.

### Ca²⁺ Buffering for CaM Measurements:

An initial CaM stock solution corresponding to 20 mg/L was made in 100 mM Tris-HCl, pH=7.0, and the subsequent CaCl₂ stock solutions were prepared as a series of concentrations corresponding to (1) 20 μM, (2) 50 μM, (3) 250 μM, (4) 1250 □μM, (5) 6250 μM, and (6) 31.25 mM. The Tris-EGTA solution contained 1 mM EGTA and 100 mM Tris-HCl, pH=7.0. The CaM solutions were freshly prepared each day according to the recipe showed in Table 3. Prior to use, CaCl₂ solutions were incubated for 20 min in order to allow the Ca²⁺ and CaM binding to equilibrate. The final concentration of CaM corresponded to 10 mg/L (0.6 μM) based on a molecular weight of 16.8 kD [see Table 3].

### Simulation of the Docking:

Some embodiments of the simulations described herein were performed with Autodock4, (a software tool provided for free by The Scripps Research Institute). As shown in equation (3), the free energy of a molecule from the van der Waals force, hydrogen bonding, electrostatics, and desolvation can be calculated as:

$$V = {W}_{vdw} {\sum }_{i , j} ( \frac{{A}_{ij}}{{r}_{ij}^{12}} - \frac{{B}_{ij}}{{r}_{ij}^{6}} ) + {W}_{hbond} {\sum }_{i , j} E ( t ) ( \frac{{C}_{ij}}{{r}_{ij}^{12}} - \frac{{D}_{ij}}{{r}_{q}^{10}} ) + {W}_{elec} {\sum }_{i , j} \frac{{q}_{i} {q}_{j}}{ɛ ( {r}_{ij} ) } + {W}_{sol} {\sum }_{i , j} ( {S}_{i} {V}_{j} + {S}_{j} {V}_{i} ) {e}^{( - {r}_{ij}^{2} / 2 {σ}^{2} )} \qquad ( 3 )$$

Here i and j indicate the atom pair, Wₓ the weighting constants, rᵢⱼ the distance between atom i and atom j, and A, B, C, and D are constant parameters. E(t) is determined by deviation from ideal hydrogen bonding geometry, depending on angle t. In the third term, q is partial atomic charges, and e is the dielectric constant. In the final term, Vₓ is the atomic fragmental volume, S is the solvation parameter, and σ is the Gaussian distance constant set at 3.5 Å.

When molecules bind, there is a changing in free energy. As expressed in equation (4), field ΔG, i.e. the free energy change, integrates the energy evaluations of molecules (Vₓ^{y}), as well as the conformational entropy lost upon binding (ΔS_{conf}).

ΔG=(V_{bound}^{L-L}−V_{unbound}^{L-L})+(V_{bound}^{P-P}−V_{unbound}^{P-P})+(V_{bound}^{P-L}−V_{unbound}^{P-L}+ΔS_{conf}) (4)

L refers to ligand, i.e. phenolic molecules, and P refers to protein.

In the computation, the root mean standard deviation (rmsd) of the docking conformations is used to measure the location similarity of dockings. Dockings within the designated rmsd limit (3.5 Å) are in the same cluster as the seed structure, while those with larger rmsd value will be used as a new seed to generate a new cluster. Protein 3D crystal structures were obtained from Protein Data Bank (PDB) and 3D structures of ligands were obtained from the ZINC and PubChem databases²⁷.

TABLE 1
The cluster amount, size and ΔG of the simulation
with CaM and the compounds.

| | |
|---|---|
| CaM-Compound 10 | CaM-Compound 6 |

| Cluster | ΔG | Cluster | ΔG | Cluster |
|---|---|---|---|---|
| number | [kcal/mol] | size | [kcal/mol] | size |
| 1 | −5.7 | 3 | −5.02 | 74 |
| 2 | −5.58 | 2 | −4.75 | 21 |
| 3 | −5.55 | 2 | −4.28 | 1 |
| 4 | −5.54 | 1 | −4.18 | 1 |
| 5 | −5.05 | 2 | −3.83 | 3 |
| 6 | −5.04 | 2 | | |
| 7 | −4.99 | 2 | | |
| 8 | −4.94 | 1 | | |
| 9 | −4.92 | 2 | | |
| 10 | −4.77 | 1 | | |
| 11 | −4.77 | 1 | | |
| 12 | −4.75 | 2 | | |
| 13 | −4.68 | 1 | | |
| 14 | −4.68 | 1 | | |
| 15 | −4.62 | 4 | | |
| 16 | −4.61 | 1 | | |
| 17 | −4.6 | 2 | | |
| 18 | −4.58 | 1 | | |
| | . | . | 70 clusters | |
| | . | . | with si | |
| | . | . | | |
| 87 | −3.55 | 1 | | |

TABLE 2
The largest binding energy ΔG [kcal/mol] between
compounds and proteins:

| | | | E7 | E6 | CaM |
|---|---|---|---|---|---|
| Compound | 1 | Phenol | −3.65 | −4.14 | −3.82 |
| | 2 | 2-(3-hydroxyphenoxy)phenol | −5.38 | −5.84 | −5.31 |
| | 3 | 2-(4-hydroxyphenoxy)phenol | −5 | −5.29 | −5.12 |
| | 4 | 3-(3-hydroxyphenoxy)phenol | −5.14 | −5.56 | −4.93 |
| | 5 | 2-(2-hydroxyphenoxy)phenol | −4.22 | −5.52 | −4.69 |
| | 6 | 4-Phenoxyphenol | −4.58 | −4.78 | −5.02 |
| | 7 | 2-Phenoxyphenol | −4.96 | −5.47 | −4.9 |
| | 8 | 3-Phenoxyphenol | −4.8 | −5.28 | −4.93 |
| | 9 | 4,4′-Dihydroxydiphenyl ether | −4.68 | −4.87 | −5.24 |
| | 10 | 2-(3,4,5- | −5.36 | −6.32 | −5.7 |
| | | trihydroxyphenoxy)benzene- | | | |
| | | 1,3,5-triol | | | |
| | 11 | 1-(2,4,6- | −5.78 | −6.73 | −5.56 |
| | | trihydroxyphenoxy)benzene- | | | |
| | | 2,3-diol | | | |
| | 12 | 3-(3-Phenoxyphenoxy)phenol | −6.73 | −6.03 | −6.15 |
| | 13 | 4,4′-(1,4- | −5.06 | −5.56 | −5.79 |
| | | phenylenebis(oxy))diphenol | | | |
| | 14 | 2,2′-[1,4- | −6.14 | −6.25 | −6.08 |
| | | phenylenebis(oxy)]diphenol | | | |
| fMer | 1 | Itaconic acid | −4.00 | −6.12 | −3.05 |
| | 2 | Acrylic acid | −3.23 | −4.61 | −2.47 |
| | 3 | 4-Divinylbenzene | −3.99 | −4.34 | −4.25 |
| | 4 | 4-VP | −3.68 | −4.31 | −3.62 |
| | 5 | MAA | −3.47 | −4.26 | −2.84 |
| | 6 | 2-Vinylpyridine | −3.77 | −4.11 | −3.64 |
| | 7 | Methylmethacrylate | −2.96 | −3.94 | −2.99 |
| | 8 | Acrylonitrile | −2.39 | −3.84 | −2.75 |
| | 9 | Acrolein | −2.05 | −3.75 | −2.34 |
| | 10 | AAM | −2.71 | −3.73 | −3.13 |
| | 11 | Allylamine | −3.03 | −3.63 | −4.46 |

TABLE 3
Recipes for Ca²⁺ buffer cocktail

| [Ca]_{free} (μM) | CaM stock (μl) | Tris-EGTA (μl) | CaCl₂ (μl) | Ca stock |
|---|---|---|---|---|
| 0 | 25 | 25 | 0 | |
| 4.14E−4 | 25 | 25 | 5 | (1) |
| 1.038E−3 | 25 | 25 | 5 | (2) |
| 5.298E−3 | 25 | 25 | 5 | (3) |
| 2.952E−2 | 25 | 25 | 5 | (4) |
| 3.439E−1 | 25 | 25 | 5 | (5) |
| 2.125E3 | 25 | 25 | 5 | (6) |

*[Ca]_{free} concentrations were calculated based on assumptions of K_{d}^{EGTA} = 207 nM.

In some further embodiments disclosed herein, high performance protein imprints were developed on nanoelectrodes. The imprinting mechanism used further computational methods to calculate binding energy and assess the molecular dynamics between compounds and the E7 biomarker of human papillomavirus.

The polymerization products showed self-assembly on the protein as bio-recognition features in the imprints. Protein biomarker takes a pivotal role in the early detection of cancers as described above. To conduct biomarker detection in biological matrices, bio-recognition components such as monoclonal antibodies and aptamers are needed to define the specific responses of nanosensors made of carbon nanotube, nanoparticle, and nanowire, etc. Also as described above, it is the performance of the bio-recognition molecule that determines the limit of detection and also governs the biological noise floor.

In some embodiments, to improve the specificity of recognition, functional monomers (fMers) are used to form pre-complex with the template. After entrapment of the complex, the fMers can be covalently fixed in the imprint structure to facilitate the recognition by binding with the target. However, it becomes complex in the practice of protein imprinting due to the massive amount of residues and flexible protein structure. In fact, protein recognition may utilize shape complementarity combined with weak van der Waals interactions and the formation of hydrogen bonds. The recognition structure is often described as interface, and atomistic molecular dynamics simulations where also used in some embodiments to analyze the assembly of molecules to identify fMers for biotoxin microcystin-LR, wherein a method of such an embodiment was used to design an imprint with specificity greater than that of a polyclonal antibody. In some embodiments, fMer screening may be performed by the computational analysis of assembly with small templates molecules.

In some embodiments, a carbon nanotube (CNT) nanoelectrode array maybe used to in situ integrate the imprint of human papillomavirus biomarker E7 protein in polyphenol (PPn) nanocoating (FIG. 8C), wherein a LOD at sub-picogram per liter level was achieved. In another embodiment the procedure of imprint fabrication was performed with a gold nanowire array for the detection of ovarian cancer biomarker CA125.14 The LOD reached to 0.5 U mL-1, which was more sensitive than ELISA. According to a mathematical model for ovarian tumor growth and the CA125 shedding the sensor of this embodiment was able to detect a tumor as small as 4 mm in diameter. Such improvements suggested arobust protein recognition by the protein imprint of FIG. 8C wherein an embodiment of the mechanism of the formation of the high performance imprint is depicted.

As such, in some embodiments during the electropolymerization step, the polymerized oligomers may assemble with the protein template. The oligomer-template complex, rather than the template molecule alone, will be entrapped in the PPn coating on the sensor, wherein the oligomers will serve as the recognition features in the imprint. In further embodiment, the electropolymerization can randomly produce oligomers with different levels of polymerization and various isomeric structures (FIG. 8B). The phenolic compounds comprise an fMer selection for the protein imprint: of either neutral or weak acidic or basic. Twelve such oligomers provide an interaction system for the proposed assembly process. In some embodiments, the protein sensor was fabricated with the human ferritin (hFtn) template.

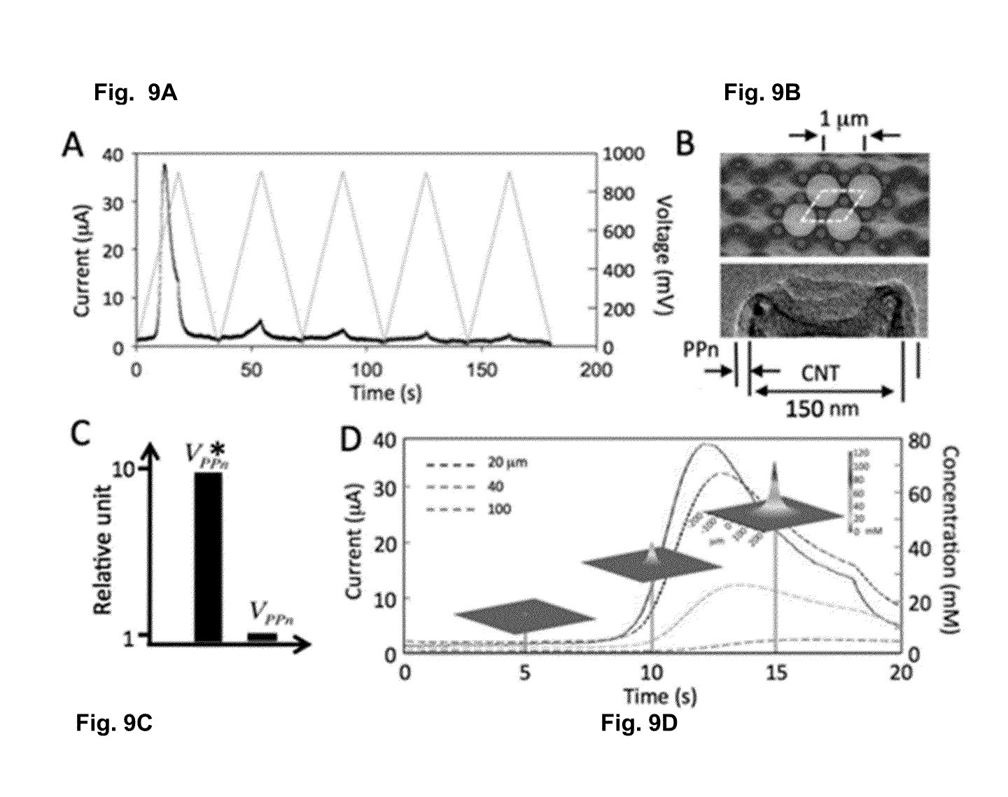

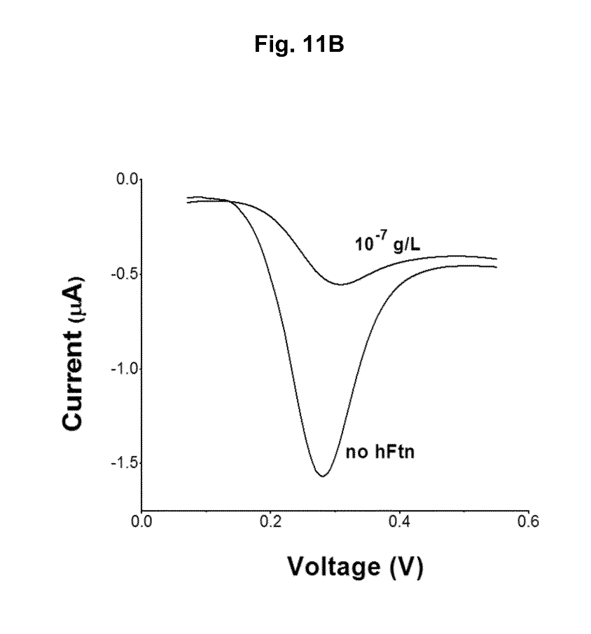

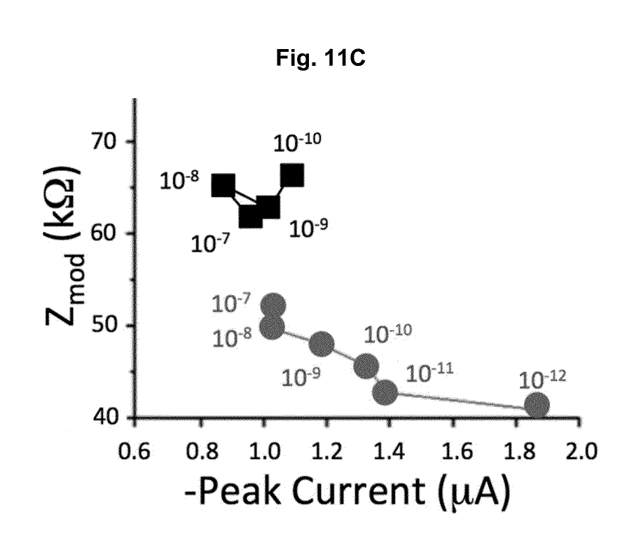

The protein detection results with an impedance and current which showed selective and sensitive responses to the hFtn target (FIG. 11(A), FIG. 11(B), FIG. 11(C)) indicating a high quality imprint because the concentration of the PPn produced in the electropolymerization is related to the assembly speed. As shown in FIG. 9, (A): in some embodiments, the electropolymerization of PPn may be self-limiting because the PPn may be non-conducting. A dominant Faradic current appeared in the first cyclic voltammetry (CV) scanning with the peak current appearing at 434 mV and producing 135 μC reaction charges, which was a major portion of all charge (146 μC) in the five voltammograms. Under transmission electron microscope (TEM), the thickness of PPn coating on CNT was 15±3 nm (n=20) (FIG. 9, (B)). Scanning electron microscopy (SEM) estimates the number of CNTs on a chip. The actual volume of PPn deposition (VPPn) was thereby estimated. On the other hand, the theoretical volume (denoted as VPPn*) was calculated according to the total charge generated in the reaction. For the PPn deposition on a planar gold electrode, the ratio k of VPPn* to VPPn is normally between 1.16 and 1.21.17. However, with the CNT nanoelectrode array as described in some embodiments, the average k value was 10.0±2 (n=20). In some further embodiments, such a value indicates that about 10% of the oxidized phenol molecules were deposited on the CNT tips, while the rest diffused into the solution (FIG. 9, (C)). The larger k values may be ascribed to the geometry of the nanosensor array that can facilitate both replenishment of analyte and prevention of the accumulation of reaction products at the electrode surface. It was reflected in the static state current as described by the Cottrell equation. According to the Faradic current, I(τ), produced in the deposition, the concentration profiles of PPn at the nanoelectrode can be calculated based on the Fick's second law of diffusion. Some embodiments show that the dimer concentration within a 20 μm distance to the electrode surface may be maintained at 60 mM during the deposition cycle (FIG. 9, (D)). The estimation is based on the conclusion that in one embodiment all PPns are dimers. To evaluate the oligomers (based on their potentials as alternative fMers) the docking information was compared. The phenolic oligomers were noted as pfMers (i.e., polyphenol fMer). Human papillomavirus E6 (PDBID:2FK4) and E7 (PDBID: 2EWL) proteins are biomarkers of human cervical cancer. They were in some embodiments used as the template proteins.

Eleven fMers were also simulated. From the docking analysis, a larger negative ΔG indicated a larger binding energy and higher affinity of pfMer-protein or fMer-protein. The largest ΔG results from 100 runs for each compound is summarized in Table 2. The oligomers in some embodiments exhibited stronger binding than did fMer molecules. The biomarkers showed different binding pfMers. For example, pfMer12 (−6.7 kcal/mol) and pfMer14 (−6.3 kcal/mol) were the strongest binding moieties for E7 and E6, respectively. By overlaying the center of mass of each pfMer on the proteins from the results of all 100 runs, some susceptible docking areas were outlined indicating the self-assembly from the energy minimization perspective. In some embodiments, All-atom MD simulation were used to investigate the self-assembly process of the pfMers. In some embodiments, the simulation system was prepared with an E7 protein molecule and 24 phenolic dimers that were solvated in a box of explicit water molecules. The ratio of E7 to the pfMers was close to that between the template and fMers in conventional imprinting. The system was equilibrated under both NVT and NPT conditions in multiple steps. In some embodiments, after the equilibration steps, the density of the system was 0.989 g/cm3 and the concentration of phenol dimer was stabilized at 69 mM. In some embodiments, three independent simulations were carried out for three phenol dimers, namely, OLS (4-Phenoxyphenol), OSE (2-Phenoxyphenol), and OET (3-Phenoxyphenol).

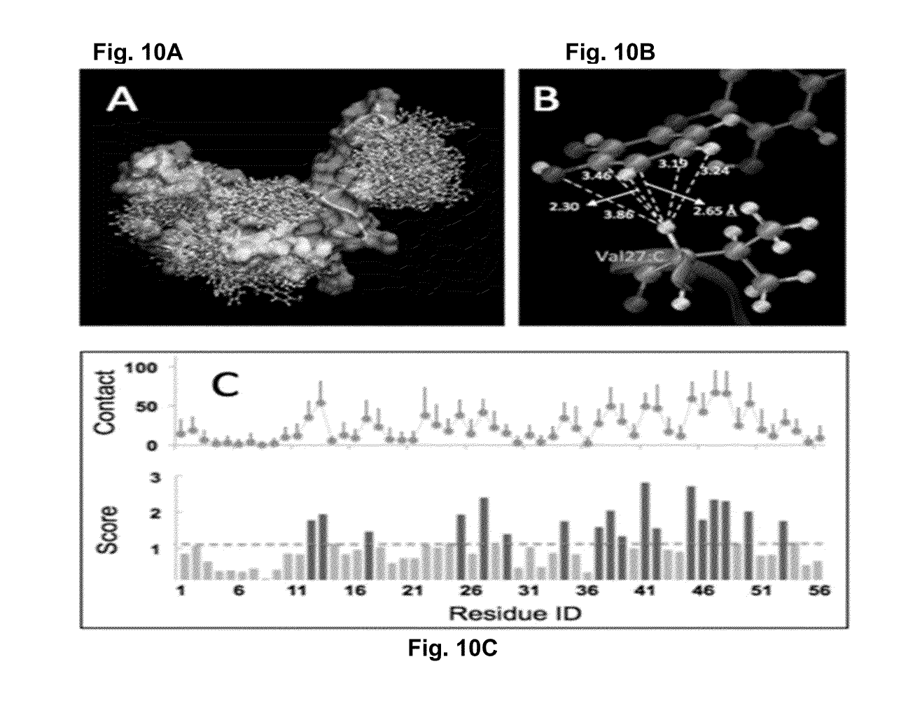

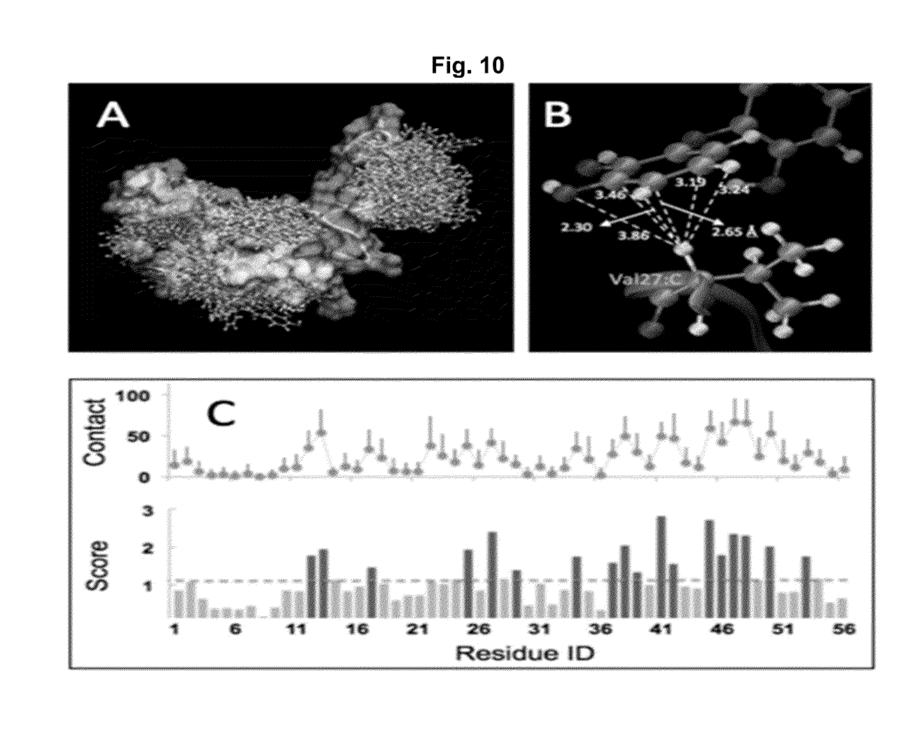

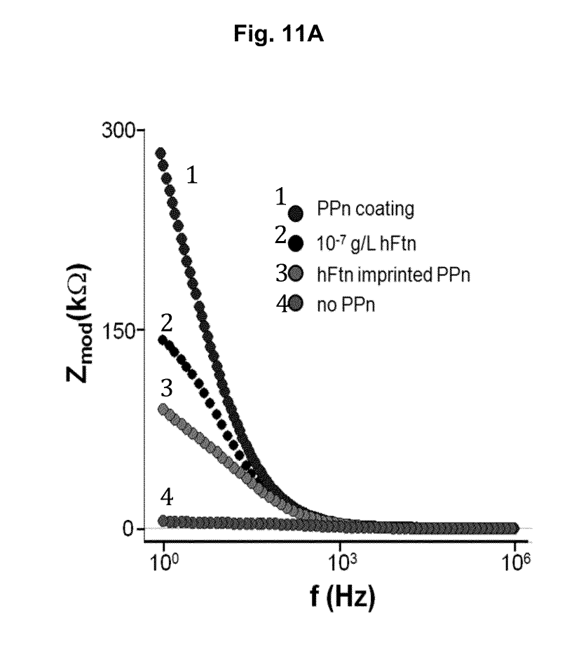

In some embodiments, association of the pfMers with the E7 protein was observed in the simulation for 100 ns (FIG. 10, (A)). The contact between the molecules was defined in FIG. 10B. If the distance between the atoms of an oligomer and a protein residue was less than 4 Angstroms (see FIG. 10, (B)), a contact was counted. The contact number was collected for each residue and averaged on the total trajectory frames. The values were largely diverged among the residues (FIG. 10, (C), top).

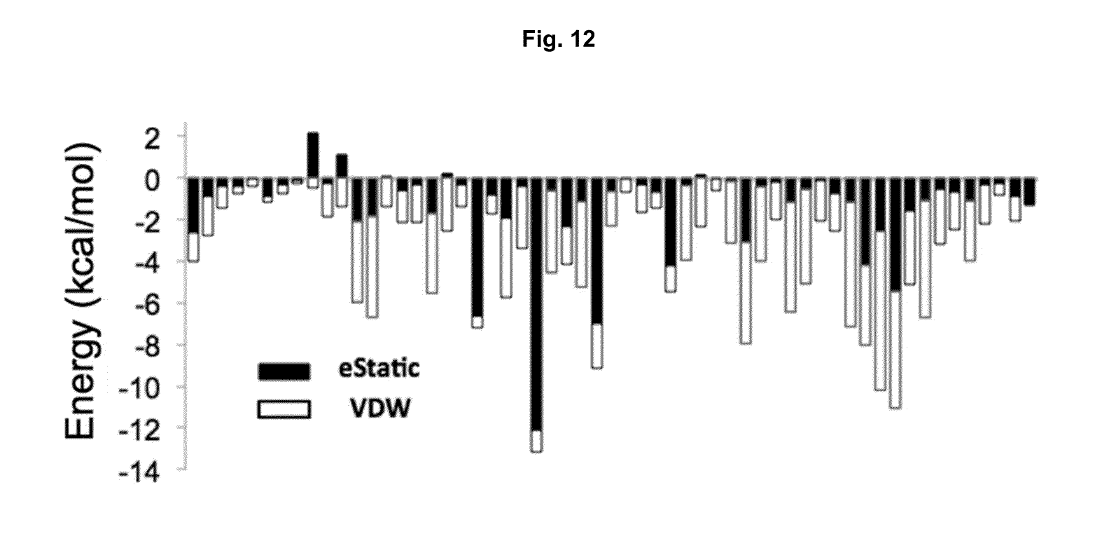

In some embodiments, the residues did not equally interact with the oligomers and scored in different levels (FIG. 10, (C), bottom). A large contact value and small deviation gave a high score, which in some embodiments indicates that the contact is stable and frequent. In the E7-OLS ensemble, the 18 high-scored and non-charged residues claimed 63% of the total contact, in which thirteen of them were hydrophobic amino acids. The electrostatic energy and the van der Waals (VDW) energy account for the contacts (FIG. 12). The negatively charged residues, i.e., 20 D, 24 E, 28 E and 33 D did not show frequent contacts, although their electrostatic energy was larger than the average. The positively charged residues have, in some embodiments, a limited amount of contacts. Just as indicated in the docking analysis, the molecular dynamic simulation described herein showed the selective interactions between OLS and the E7 protein residues

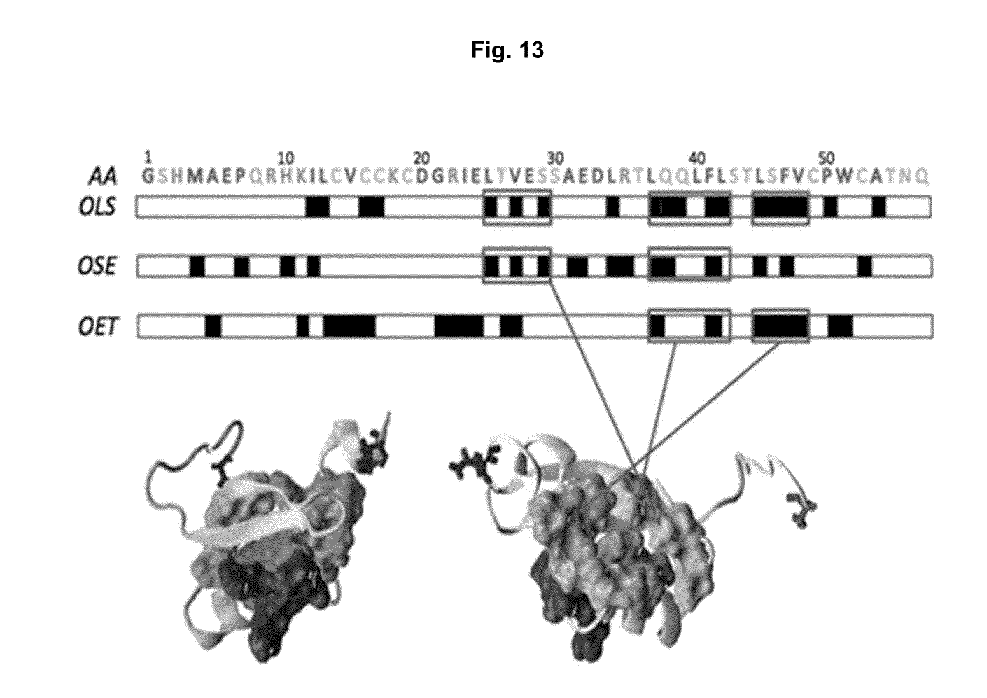

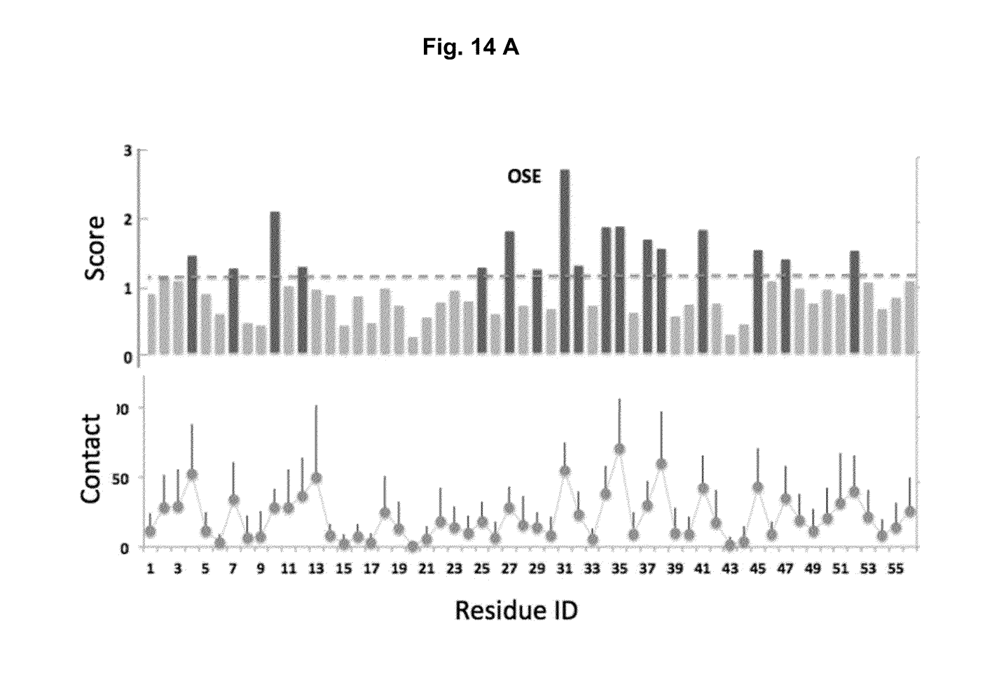

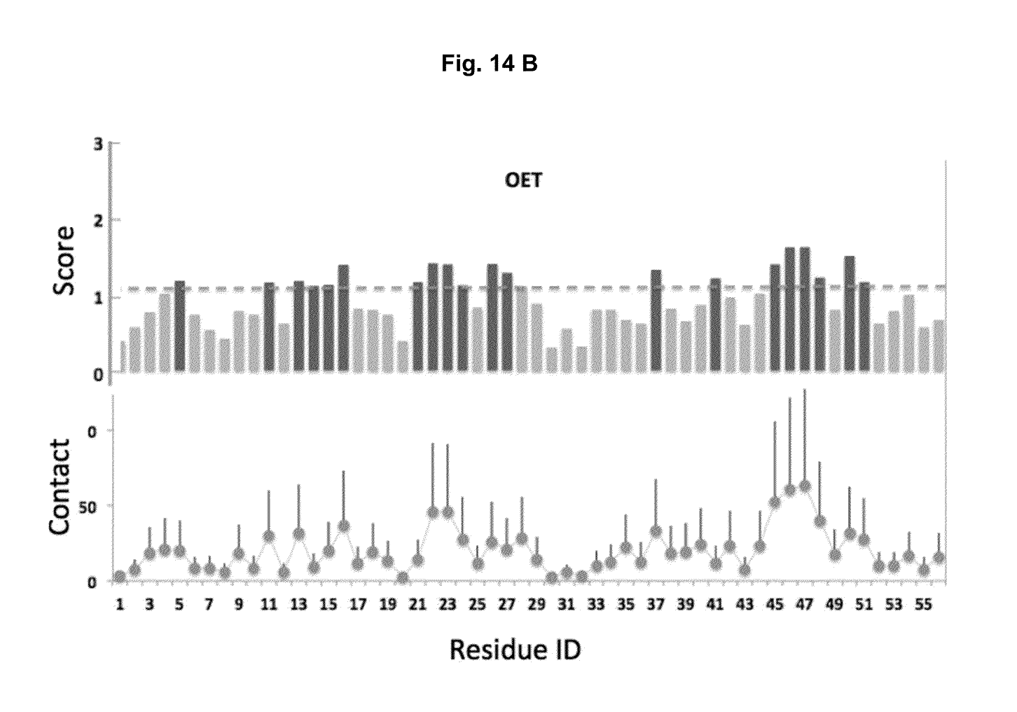

In some further embodiments, similar results were also observed in the simulations of two other oligomers, OSE and OET. However, they were not identical, as compared in FIGS. 14(a) and (b). The high-score (i.e., score>=1.2) residues for the three oligomers were collected in FIG. 13. The hydrophobic residues claimed 72, 65, and 65% of the contact with OLS, OSE, and OET, respectively. OLS showed three highly frequent binding regions, i.e., R1: (25, 27, 29), R2: (45, 46, 47, 48), and R3 (37, 42), which were consensus with OSE in R1 and R3, and OET in R2 and R3. In fact, all those grouped residues were continuous patches of a contact interface.

In the electropolymerization process, the interaction with template proteins may take place with a mixture of the pfMers. In some embodiments of the mixtures, the pfMers may compete for binding with the template. To characterize the details, a competition system containing 12 OLS, 12 OET and an E7 protein in a water filled box were set up. After the same equilibration period as previously described, the simulation was run for 100 ns with NAMD.

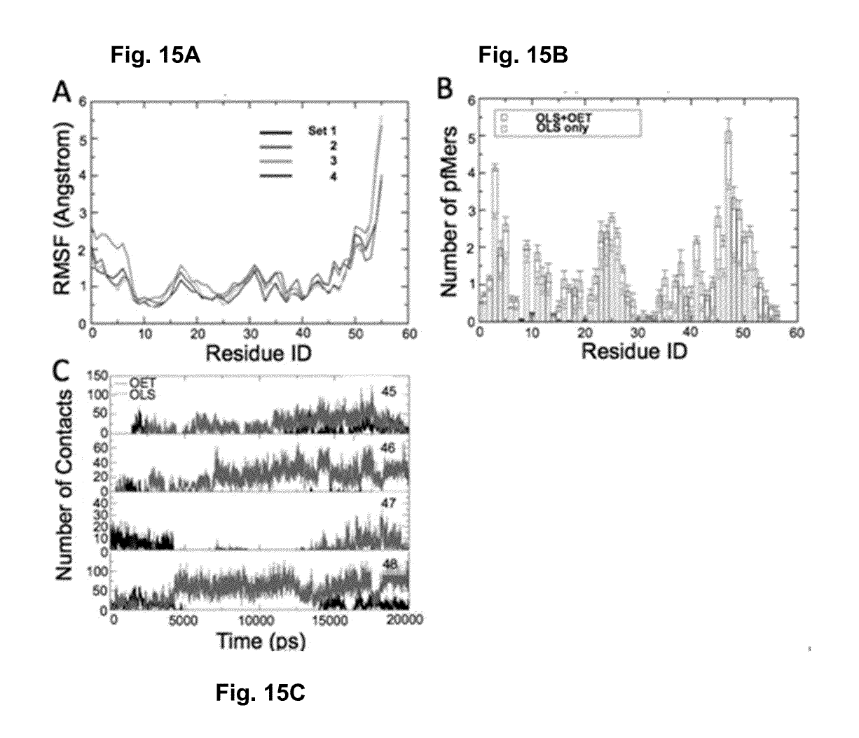

Four sets of follow-up 20 ns NAMD simulation initiated with the same trajectory from the end of the 100 ns were conducted to evaluate the convergence of the assembly were then performed. Root mean squared fluctuation (RMSF) for the backbone only was calculated to quantitatively measure the magnitude of the deviation between atomic positions of residues and their native structure from the trajectory. It showed large magnitudes of fluctuation at both terminals (FIG. 15, (A)). The residues between 6 and 50 were in some embodiments stable with their RMSF values stayed below 3.6 Angstroms for the first 20 ns (data not shown) and 2.5 Angstroms for the 20 ns following the end of 100 ns.

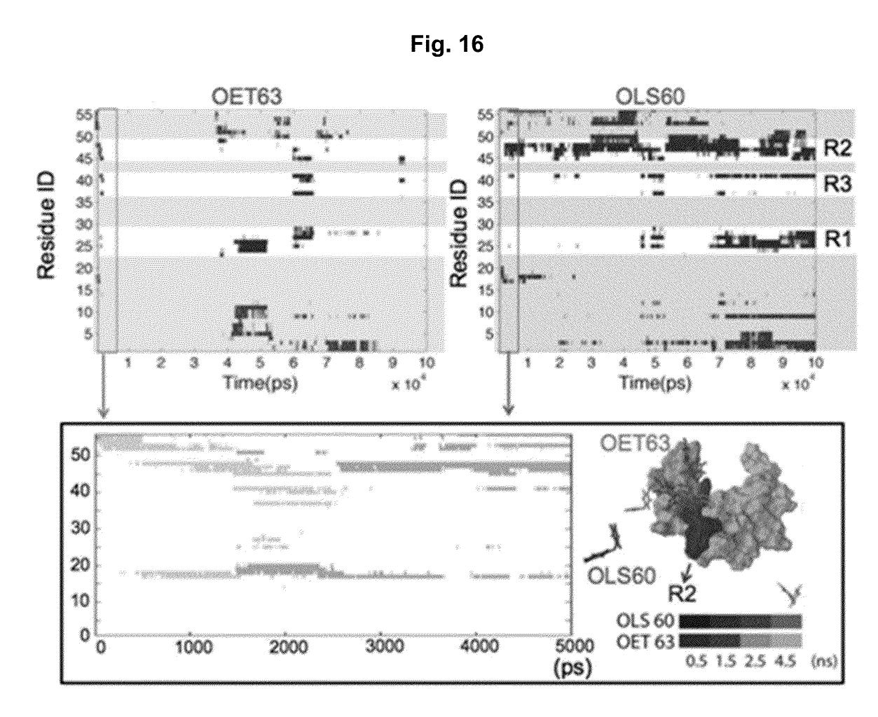

FIG. 15, (B) showed statistically the amount of pfMers contacting with the residues per frame. The simulation indicated that OLS dominated the pfMers' competition for the assembly with E7, since it contributed to 60±4% of the total contacts. Considering a sequence with residues from 6 to 50, the high contact frequency was observed in two major motifs: residue 20 and 30, and residue 40 and 50. They matched the contact regions that OLS shared with OSE and OET, respectively (FIG. 13). Hence, the contact pattern resulting from the competition resembled that from the simulation of OLS only. The structural similarity in the pfMers largely minimized the energy barrier for OLS to interfere with OET's own binding activity and take over the occupancy of binding site. As shown in FIG. 15, (C), OET pfMers occupied the four residues of R2:(45,46,47,48) at beginning. OLS pfMers competed for the binding and replaced OETs after 5 ns. The competition was also viewed from the trajectory of the oligomers. As shown in FIG. 16, the contacts with residues by OET 63 (pfMer OET with molecular ID 63 in the system) and OLS60 in 100 ns were plotted. OET63 got in contact with E7 at multiple motifs including R1, R2 and R3 earlier than OLS60. At 3.5 ns, OET63 completely lost contact with the E7, but OLS60 stayed on. At 41 ns, OET63 came back and occupied R1. However, in some embodiments, it lost the contact at 50 ns. In further embodiments, OET63 did not retain its contact at any part of the E7 longer than 10 ns, while OLS60 secured the contacts R1 and R3 for 30 ns and R2 for almost 100 ns.

Hence, in some embodiments, the results herein described show that docking and molecular dynamics methods may be used in combination to evaluate the interaction between the fMer candidates and the template protein. The contact score is a useful parameter to identify the contact regions of the fMer-protein assembly. The competition analysis may be an alternative verification of the contact region results. In addition to the traditional understanding of the electropolymerization in imprinting, it is suggested that an in situ self-assembly process could be mediated by the nanoelectrodes to derive bio-recognition features in the imprints.

As used in some further embodiments described herein, the following methods and materials were used and comprised the following Reagents and instrumentation: Phenol, phenol oligomers, ferrocene carboxyl acid (FCA) and bovine serum albumin were obtained from Sigma-Aldrich (St. Louis, Mo.). Human ferritin protein was obtained from AbDSerotec (Raleigh, N.C.). Horse ferritin and apoferritin were obtained from MP Biomedicals (Solon, Ohio). Phosphate buffered saline was obtained from Fisher Scientific (Pittsburgh, Pa.). Electropolymerization and electrochemical behavior of thin-films were conducted with a Reference 600 electrochemical system supplied by Gamry Inc. (Warminster, Pa.) operating via Framework. Data analysis was conducted with Echem Analyst. TEM images of PPn coated CNTs were obtained with a JEM-2010F TEM (JEOL, Tokyo, Japan) operating at 200 kV.

CNT array preparation: In some embodiments, array fabrication begins with growth CNT^{[13]} via plasma-enhanced chemical vapor deposition on polystyrene sphere-patterned substrates, followed by embedding in SU8-2002 photoresist and then mechanically polished to expose the tips. First, SU8 was spun on an array at 3000 rpm for 30 s. Following a soft bake for 5 min at 100° C., SU8 was cross-linked by exposure to UV light for 3 min and then the sample was incubated at 150° C. overnight. Lastly, the chip was polished with a vibratory polisher (Buehler in Lake Bluff, Ill.) with 80% power level for 6-9 hrs until the pattern was revealed.

Electrochemistry Experiments:

In some embodiments, a three-electrode electrochemical system included: the CNT chip, chlorinated silver wire, and platinum wire as the working, reference and counter electrodes, respectively. PPn film was deposited on the CNT array by cyclic voltammetry in a phenol (1.5 mM) supplemented phosphate buffered saline (PBS), pH=7.4. The potential on the working electrode was scanned 5 times between 0 to 0.9 V vs. the reference electrode. In order to entrap ferritin in the PPn coating, the protein (100 μg/ml) was added to the PPn deposition buffer. Following electrophoretic attraction by applying 300 mV DC voltage for 30 s, cyclic voltammetric voltages were used to form the PPn coating as described above for co-deposition of ferritin. EIS was conducted before and after the PPn deposition to evaluate the impedance properties of the electrode surface and its interface to the buffer solution containing FCA (1 mM) in PBS. The sine wave was 10 mV peak-to-peak in amplitude. It was superimposed on a 300 mV DC voltage. Frequency was scanned from 1 Hz to 1 MHz. The impedance data were fitted to an electrical equivalent circuit using the impedance analysis function in Echem Analyst software. DPV was conducted in the same buffer as that for EIS. Initial and final potentials vs. reference electrode were 0 and 0.5 V respectively. Pulse size was 50 mV and pulse time was 0.05 s; step size was 2 mV, and sample period was 0.1 s. Protein imprint development: In some embodiments, for imprint development, the sensor with ferritin-entrapped PPn coating was rinsed and incubated overnight in deionized water at room temperature. Alternatively, a developing buffer containing acetic acid (5% w/v) and sodium dodecyl sulfate (SDS) (10% w/v) was used for higher protein extract efficiency. After ferritin entrapment and removal, the sample was evaluated by TEM and EIS. To prepare TEM samples, coated CNTs were carefully scraped off with a sharp blade in isopropanol. About 10 μl of CNT suspension was dropped onto TEM copper grids with carbon film. The samples were checked with TEM immediately after isopropanol was vaporized. With this specimen preparation, the cross-section of the coated CNTs could be observed by TEM bright field imaging.

Molecular Dynamic Simulation with NAMD (NAnoscale Molecular Dynamics Program):

System Preparation. In some embodiments, Molecular Dynamics simulations were performed for E7 protein in explicit water molecules with oligomers. Three independent simulations were prepared for each type of oligomers—OLS, OLE, and OLT. In each system, initially oligomers were randomly placed around E7 protein at a distance of 15 Å or greater from the surface of the protein, or from each other. For the simulation of pfMer competition, 12 OET and 12 OLS were added into the system with random position The structural Hamiltonian and the parameters of the Lennard-Jones potential for the oligomers were taken from the General Amber Force Field (GAFF) and Antechamber from AMBER10. The point charges for the oligomers were obtained with the AM1-BCC method as implemented in the Antechamber. Water was modeled by TIP3P model and E7 protein was modeled by Amber force field 99SB. The simulations were carried out with NAMD.

MD Equilibration During the simulations, the bonds were fixed by using the SHAKE algorithm with the integration time step of 2 fs. The oscillation decay time for the Langevin dynamics was 50 fs. The switching distance on the Lennard-Jones term was set at 10 Å and the cutoff was 12 Å. The electrostatics interactions were calculated using the particle mesh Ewald (PME) method with a grid size less than 1/Å³ spacing. The protein-oligomers-water system was equilibrated as follows: Initially, the water molecules were energetically minimized using the conjugate gradient method, and then gradually heated to 300 K with a temperature step of 3 K. At each temperature step, the solvent molecules were equilibrated for 2 ps under a constant NVT conditions using Langevin dynamics. After the final temperature of 300 K was achieved, the solvent molecules were equilibrated further for 5 ns under constant NVT. To adjust the density of water, an equilibration of 5 ns under constant NPT conditions was performed using the Note-Hoover Langevin piston pressure control. At this stage, the density of protein-oligomer-water system was close to the liquid water density at 300 K, followed by another 10 ns under a constant NVT. From the well-equilibrated system, the production runs were performed for a total of 100 ns. In the competition simulation, a100 ns NAMD simulation was performed first, then 4 sets of follow-up 20 ns NAMD simulation, which restart from the end of previous 100 ns simulation, was performed.

Calculation:

The actual volume of PPn (V_{PPn}) can be calculated according to following equation:

V_{PPn}=N·V (1)

N is the total amount of nanotubes on the sensor chip. V is the PPn volume on each nanotube. For the example in FIG. 9C, N=6.24×10⁷, V=2.65×10⁻²² m³. Accordingly, we have V_{PPn}=1.7×10⁻¹⁴ m³.

Given all phenol participated in the reaction are deposited on the CNT, we can have an alternative way to get the volume of PPn, (V_{PPn}*):

$${V}_{PPn}^{*} = \frac{m \cdot {Q}_{PPn}}{F \cdot σ} \qquad ( 2 )$$

m is 94 g/mole, Q_{PPn} is the total charge generated during the electropolymerization and equals to 146° C. according to FIG. 9 (A-D). F is the Faradic constant, 9.65×10⁴ C/mole. σ□ is the density of phenol, 10⁶ g/m³.

Calculated with all numbers above, we have V_{PPn}*=1.42×10⁻¹³ m³. Therefore, in the example shown in FIG. 9(A-D),

V_{PPn}=(1/8.3)·V*_{PPn}

References that are cited herein are incorporated by reference herein in their entirety. While exemplary embodiments have been shown and described, modifications thereof can be made by one skilled in the art without departing from the scope or teachings of this disclosure. The embodiments described herein are exemplary only and are not limiting. Many variations and modifications of the systems, apparatus, and processes described herein are possible and are within the scope of the disclosure. Accordingly, the scope of protection is not limited to the exemplary embodiments described herein, but is only limited by the claims that follow, the scope of which shall include all equivalents of the subject matter of the claims.
[1] J. A. Ludwig, J. N. Weinstein, Nat. Rev. Cancer, 2005, 11, 845-856
[2] M. F. Ullah, M. Aatif, Cancer Treat Rev., 2009, 3, 193-200.
[3] A. Warsinke, Anal Bioanal, 2009, 393, 1393-1405.
[4] http://www.wired.com/medtech/health/magazine/17-01/ff_cancer?currentPage=all
[5] Guidelines for the validation and verification of quantitative and qualitative test methods, National Association of Testing Authorities, Australia, 2012.
[6] J. A. Ho, H. C. Chang, N. Y. Shih, L. C. Wu, Y. F. Chang, C. C. Chen, C. Chou, Anal. Chem., 2010, 82, 5944-5950.
[7] Q. Wei, Y. Zhao, C. Xu, D. Wu, Y. Cai, J. He, Li, B. Du, M. Yang, Biosens. Bioelectron., 2011, 26, 3714-3718.
[8] S. Viswanathan, C. Rani, A. V. Anand, J. A. Ho, Biosens. Bioelectron., 2009, 24, 1984-1989.
[9] J. Lin, Z. Wei, C. Mao, Biosens. Bioelectron., 2011, 29, 40-45.
[10] A. M. Ionescu, Nat. Nanotechnol., 2010, 5, 178-179.
[11] V. Mani, B. V. Chikkaveeraiah, V. Patel, J. S. Gutkind, J. F. Rusling, ACS Nano, 2009, 3, 585-594.
[12] D. M. Rissin, C. W. Kan, T. G. Campbell, S. C. Howes, D. R. Fournier, L. Song, T. Piech, P. P. Patel, L. Chang, A. J. Rivnak, E. P. Ferrell, J. D. Randall, G. K. Provuncher, D. R. Walt, D. C. Duffy, Nat. Biotechnol., 2010, 28, 595-599.
[13] J. Das, S. O. Kelley, Anal. Chem., 2011, 83, 1167-1172.
[14] T. Takeuchi, T. Hishiya, Org. Biomol. Chem., 2008, 6, 2459-2467.
[16] M. J. Whitcombe, I. Chianella, L. Larcombe, S. A. Piletsky, J. Noble, R. Porter, A. Horgan, Chem. Soc. Rev., 2011, 40, 1547-1571.
[17] J. Wang, S. Yau, Biosens. Bioelectron., 2011, 29, 210-214.
[18] Y. Li, Y Li, M. Hong, Q. Bin, Z. Lin, Z. Lin, Z. Cai, G. Chen, Biosens. Bioelectron., 2013, 15, 612-617.
[19] D. Cai, L. Ren, H. Zhao, C. Xu, L. Zhang, Y. Yu, H. Wang, Y. Lan, M. F. Roberts, J. Chuang, M. J. Naughton, Z. Ren, T. C. Chiles, Nature Nanotech. 2010, 5, 597-601.
[20] S. Viswanathan, C. Rani, S. Ribeiro, C. Delerue-Matos, Biosens. Bioelectron., 2012, 33, 179-183.
[22] A. M. Lutz, J. K. Willmann, F. V. Cochran, P. Ray, S. S. Gambhir, PLoS Med., 2008, 19, e170.
[23] M. J. Whitcombe, D. Lakshmi, Electropolymerization: Concepts, Materials and Applications, Eds. Wiley, 2010.
[25] A. J. Bard, L. R. Faulkner, Electrochemical Methods: Fundamentals and Applications, John Wiley & Sons Inc., 2000, 161-174.
[26] M. A. Vorotyntsev, V. A. Zinovyeva, D. V. Konev, Electropolymerization: Concepts, Materials and Applications, Eds., Wiley, 2010.
[27] J. Wang, M. Jiang, F. Lu, J. Electroanal. Chem., 1998, 444, 127-132.
[28] Z. Ezerskis, G. Stalnionis, Z. Jusys, J. Appl. Electrochem., 2002, 32, 49; F. Bruno, M. C. Pham, J. E. Dubois, Electrochimica Acta, 1977, 22, 451-457.
[29] P. J. Floryin, Principles of Polymer Chemistry, Cornell University Press, 1953.
[1] J. A. Ludwig, J. N. Weinstein, Nat. Rev. Cancer, 2005, 11, 845-856
[2] M. F. Ullah, M. Aatif, Cancer Treat Rev., 2009, 3, 193-200.
[3] A. Warsinke, Anal Bioanal, 2009, 393, 1393-1405.
[4] http://www.wired.com/meditech/health/magazine/17-01/ff_cancer?currentPage=all
[5] Guidelines for the validation and verification of quantitative and qualitative test methods, National Association of Testing Authorities, Australia, 2012.
[6] J. A. Ho, H. C. Chang, N. Y. Shih, L. C. Wu, Y. F. Chang, C. C. Chen, C. Chou, Anal. Chem., 2010, 82, 5944-5950.
[7] Q. Wei, Y. Zhao, C. Xu, D. Wu, Y. Cai, J. He, Li, B. Du, M. Yang, Biosens. Bioelectron., 2011, 26, 3714-3718.
[8] S. Viswanathan, C. Rani, A. V. Anand, J. A. Ho, Biosens. Bioelectron., 2009, 24, 1984-1989.
[9] J. Lin, Z. Wei, C. Mao, Biosens. Bioelectron., 2011, 29, 40-45.
[10] A. M. Ionescu, Nat. Nanotechnol., 2010, 5, 178-179.
[11] V. Mani, B. V. Chikkaveeraiah, V. Patel, J. S. Gutkind, J. F. Rusling, ACS Nano, 2009, 3, 585-594.
[12] D. M. Rissin, C. W. Kan, T. G. Campbell, S. C. Howes, D. R. Fournier, L. Song, T. Piech, P. P. Patel, L. Chang, A. J. Rivnak, E. P. Ferrell, J. D. Randall, G. K. Provuncher, D. R. Walt, D. C. Duffy, Nat. Biotechnol., 2010, 28, 595-599.
[13] J. Das, S. O. Kelley, Anal. Chem., 2011, 83, 1167-1172.
[14] T. Takeuchi, T. Hishiya, Org. Biomol. Chem., 2008, 6, 2459-2467.
[16] M. J. Whitcombe, I. Chianella, L. Larcombe, S. A. Piletsky, J. Noble, R. Porter, A. Horgan, Chem. Soc. Rev., 2011, 40, 1547-1571.
[17] J. Wang, S. Yau, Biosens. Bioelectron., 2011, 29, 210-214.
[18] Y. Li, Y Li, M. Hong, Q. Bin, Z. Un, Z. Lin, Z. Cai, G. Chen, Biosens. Bioelectron., 2013, 15, 612-617.
[19] D. Cai, L. Ren, H. Zhao, C. Xu, L. Zhang, Y. Yu, H. Wang, Y. Lan, M. F. Roberts, J. Chuang, M. J. Naughton, Z. Ren, T. C. Chiles, Nature Nanotech. 2010, 5, 597-601.
[20] S. Viswanathan, C. Rani, S. Ribeiro, C. Delerue-Matos, Biosens. Bioelectron., 2012, 33, 179-183.
[22] A. M. Lutz, J. K. Willmann, F. V. Cochran, P. Ray, S. S. Gambhir, PLoS Med., 2008, 19, e170.
[23] M. J. Whitcombe, D. Lakshmi, Electropolymerization: Concepts, Materials and Applications, Eds. Wiley, 2010.
[25] A. J. Bard, L. R. Faulkner, Electrochemical Methods: Fundamentals and Applications, John Wiley & Sons Inc., 2000, 161-174.
[26] M. A. Vorotyntsev, V. A. Zinovyeva, D. V. Konev, Electropolymerization: Concepts, Materials and Applications, Eds., Wiley, 2010.
[27] J. Wang, M. Jiang, F. Lu, J. Electroanal. Chem., 1998, 444, 127-132.
[28] Z. Ezerskis, G. Stalnionis, Z. Jusys, J. Appl. Electrochem., 2002, 32, 49; F. Bruno, M. C. Pham, J. E. Dubois, Electrochimica Acta, 1977, 22, 451-457.
[29] P. J. Floryin, Principles of Polymer Chemistry, Cornell University Press, 1953.

What is claimed is:

1. A bio-recognition sensor comprising:
(a) a polymer scaffold comprising an electrode, and an electropolymer, wherein said electropolymer forms a coating on said electrode; and
(b) a template, wherein said template comprises:
a three dimensional imprint of a biological structure in said electropolymer;
wherein said template comprises chemical moieties that are selective for binding to said biological structure.

2. The bio-recognition sensor of claim 1, wherein said electrode is comprised of carbon nanotubes.

3. The bio-recognition sensor of claim 1, wherein said electropolymer comprises polyphenol.

4. The bio-recognition sensor of claim 1, wherein said electropolymer comprises a non-conductive electropolymerized polymer.

5. The bio-recognition sensor of claim 1, wherein said non-conductive electropolymerized polymer comprises 3-aminophenol, 3-methyphenol, 3-nitrophenol, 1,3-dihydoxybenzene, 1,2-dihydroxybenzene, 1,4-dihydroxybenzene or a combination thereof.

6. The bio-recognition sensor of claim 1, wherein said coating is about 15 nm in thickness.

7. The bio-recognition sensor of claim 1, wherein said chemical moieties are comprised of oxidation species of said electropolymer.

8. The bio-recognition sensor of claim 7, wherein said oxidation species comprise ortho, meta and para phenolic monomers, dimers and trimers.

9. A method of making a bio-recognition sensor comprising:
(a) forming a electropolymer coating on an electrode;
(b) binding a target biological structure to said coating to form an imprint;
(c) removing said biological structure from said coating; and
(d) forming a template; wherein said template binds a specific biological structure.

10. The method of making a bio-recognition sensor of claim 9, further comprising:
adding pre-complexation moieties after step (a) and prior to step (b), wherein said pre-complexation moieties bind to the biological structure of step (b).

11. A method of making a bio-recognition sensor comprising:
(a) forming a electropolymer coating on an electrode;
(b) adding pre-complexation chemical moieties;
(c) adding a target biological structure;
(d) binding the pre-complexation chemical moiety to the target biological structure;
(e) binding the target biological structure comprising the pre-complexation chemical moiety to said coating to form an imprint;
(f) removing said biological structure from said coating;
(g) retaining the pre-complexation chemical moiety bound to the electropolymer coating; and
(h) forming a template, wherein said template comprises specific chemical binding moieties selective for said biological structure.

12. The method of claim 11, wherein said pre-complexation chemical moieties comprise chemical compounds with large free energy of binding for the biological structure.

13. The method of claim 12, wherein said pre-complexation chemical moieties are identified by:
docking a library of small molecules to said biological structure in silico;
identifying compound clusters, wherein said compound clusters are docked at a specific binding motif; and
calculating a free energy of binding of a representative compound of each said cluster; wherein said compound with the greatest change in free energy of binding to said biological structure is selected as a pre-complexation chemical moiety.

14. The method of claim 13, wherein said pre-complexation chemical moieties are further identified by:
calculating a free energy of binding of a representative compound of each said cluster; wherein said compound with the greatest change in free energy of binding to said biological structure is selected as a pre-complexation chemical moiety; and
verifying the binding by molecular dynamics, wherein said verifying (1) extracts the binding interfaces and scores the binding of the chemical moieties.

15. The biological structure of claim 11, wherein said structure is a native protein, a protein comprising a mutation, a protein comprising a post-translational modification, a molecule; or a peptide.

16. The bio-recognition sensor of claim 11, wherein said electropolymer comprises a non-conducting electropolymer, wherein said polymer comprises 3-aminophenol, 3-methyphenol, 3-nitrophenol, 1,3-dihydoxybenzene, 1,2-dihydroxybenzene, 1,4-dihydroxybenzene or a combination thereof.

17. The method of claim 11, wherein said biological structure is a biomarker indicative of a disease.

18. The method of claim 11, wherein said sample comprises a biological sample from a subject in need thereof of detection of a biomarker for a disease state, grade, or subclass.

19. The method of claim 18, wherein said detection of said biomarker for said disease aids in selection of a therapeutic for a disease treatment.

20. The method of claim 18, wherein said sample comprises a biological sample from a subject in need thereof of detection of a biomarker for the prognosis of a disease.
